(19) United States
(12) Patent Application Publication
(10) Pub. No.: US 2026/0182913 A1
(43) Pub. Date: Jul. 2, 2026

(54) MONITORING OF DISEASE RESPONSE TO TREATMENT

(71) Applicant: Breast-Med, Inc., Plymouth, MN (US)

(72) Inventor: Michael T. Nelson, Plymouth, MN (US)

(21) Appl. No.: 19/544,607

(22) Filed: Feb. 19, 2026

Related U.S. Application Data
(63) Continuation-in-part of application No. PCT/US2024/042922, filed on Aug. 19, 2024.
(60) Provisional application No. 63/520,613, filed on Aug. 19, 2023.

(51) Int. Cl. A61B 5/00; A61B 5/01; G16H 10/60; G16H 50/70

(52) U.S. Cl. CPC A61B 5/4848; A61B 5/01; A61B 5/4842; A61B 5/6867; A61B 5/7267; A61B 5/742; G16H 10/60; G16H 50/70; A61B 2562/0271

(57) ABSTRACT

In an example, one or more implanted tumor temperature sensors may be used in a method to determine an oncological status metric for a patient having one or more cancerous tumors. The tumor temperature measurements are collected, along with a type of cancer and one or more descriptors of a cancer treatment regimen, to calculate the oncological status metric. The oncological status metric is an indicator of cancer treatment efficacy in a patient and may be determined in part, by a programmed algorithm or trained machine learning model.

### CLAIM OF PRIORITY

This application is a continuation-in-part of and claims the benefit of priority of PCT Patent Application No. PCT/US2024/042922, entitled MONITORING OF DISEASE RESPONSE TO TREATMENT, (which was filed on Aug. 19, 2024, Attorney Docket No. 2809.007WO1), through which priority is also claimed to U.S. Provisional Patent Application No. 63/520,613 entitled TUMOR THERAPEUTIC RESPONSE MONITORED BY TELEMETRIC TEMPERATURE SENSING, (which was filed on Aug. 30, 2023, Attorney Docket No. 2809.007PRV), the priority of each of which is claimed, and each of which are hereby incorporated herein by reference in their entireties.

### TECHNICAL FIELD

This document pertains generally, but not by way of limitation, to monitoring of cancer patient response to oncological treatment.

### BACKGROUND

In oncology, treatment may be delivered in an individual treatment session, or in multiple treatment sessions making up a course of treatment. Oncological status, including response to treatment, may be monitored during or after the course of treatment.

### SUMMARY

The present disclosure recognizes, among other things, that a problem to be solved can include determining an efficacy of an oncological treatment for a cancer patient. The present subject matter can help provide a solution to this problem, such as by monitoring response of a tumor temperature to one or more oncological treatments or a course of treatment. For example, one or more sensors may be implanted in a patient. The sensors may facilitate determining an oncological status metric during or over a course of treatments of the patient. In an example, the one or more sensors can measure tumor temperature of the tumor in the patient.

The tumor temperature may change in response to treatment of the cancerous tissue (tumor). A change in the tumor temperature may indicate whether a treatment is effective against the cancer. For example, a sensor configured for measuring tumor temperature may be implanted in association with the tumor tissue, for instance implanted within the tumor of the patient. A treatment may be applied to the patient, for example to treat the cancerous tissue of the patient. The tumor temperature may be measured before cancer treatment. The tumor temperature may be measured after cancer treatment. The tumor temperature before cancer treatment may be compared to the tumor temperature after cancer treatment, such as to determine a change in the tumor temperature in response to the cancer treatment. In another example, the tumor temperature can be monitored after cancer treatment, for example at a first time after cancer treatment and a second time after cancer treatment or at multiple times after cancer treatment, including real time where measurements could be continuously for a period of time. The tumor temperature at the first time after cancer treatment may be compared with the tumor temperature at the second time after cancer treatment or continuous measurements after treatment to determine the change in tumor temperature in response to cancer treatment at one or more times after one or more treatments. Accordingly, the method to determine an oncological status metric can facilitate monitoring of tumor temperature in response to treatment of a cancer. Thus, efficacy of the treatment against the cancer may be ascertained based on the oncological status metric, which can be calculated using two or more tumor temperature measurements.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 shows a schematic diagram of an example of a device for measuring one or more physiological characteristics of a subject.

FIG. 2 shows a schematic diagram of an example of a first device and a second device implanted in the subject.

FIG. 3 shows a schematic diagram of a physiological characteristic monitoring system.

FIG. 4 shows a flowchart of a method of using one or more human-implanted or animal-implanted devices for determining medical diagnostic information over a course of treatment of a human or animal subject.

FIG. 5 shows a flowchart of an example of a computer-implemented method for monitoring a subject of a medical treatment.

FIG. 6 shows a schematic diagram of another example of the system of FIG. 3.

FIG. 7 shows a block diagram of an example of a machine upon which any one or more of the techniques discussed herein may be performed.

FIGS. 8A-8C show schematics images of an exemplary temperature monitoring study in mice, using exemplary temperature sensors implanted into tumor-bearing mice.

FIGS. 9A-9C show exemplary temperature sensor data collected from tumor bearing mice.

FIGS. 10A-10C show exemplary temperature sensor data collected from tumor bearing mice.

FIG. 11 shows a schematic depicting an SEM path analysis for temperature data collected from an exemplary temperature sensor.

FIG. 12 shows a schematic depicting the theoretical repeatability impact distribution of an exemplary 400 kHz temperature sensor.

FIG. 13 shows a schematic depicting the theoretical repeatability impact distribution of an exemplary 134.2 kHz temperature sensor.

FIG. 14 shows graphs depicting the uncertainty corrections of an exemplary temperature sensor using a kernel density function, for the observed temperatures from each treatment arm in an exemplary in vivo mouse study.

FIG. 15 shows graphs depicting the uncertainty corrections of an exemplary temperature sensor using a normal density function, for the observed temperatures from each treatment arm in an exemplary in vivo mouse study.

FIG. 16 shows graphs depicting the uncertainty corrections of an exemplary temperature sensor using a kernel density function, for the observed temperatures from the combined treatment arms in an exemplary in vivo mouse study.

FIG. 17 shows graphs depicting the uncertainty corrections of an exemplary temperature sensor using a normal density function, for the observed temperatures from the combined treatment arms in an exemplary in vivo mouse study.

FIG. 18 shows graphs depicting the temperature change distribution analysis for tumor treatment group by body location from an exemplary in vivo mouse study.

FIG. 19 shows a graph depicting the distributions of temperature differences between tumor treatment groups from an exemplary in vivo mouse study.

FIG. 20 shows a graph depicting temperature differential data over time and as a function of treatment group from tumor-bearing mice in an exemplary in vivo mouse study.

FIG. 21 shows radial smoothing graphs depicting the effect of treatment on temperature differentials in tumor-bearing mice in an exemplary in vivo mouse study.

FIG. 22 shows graphs depicting the temperature change distribution analysis for tumor treatment group by body location from an exemplary in vivo mouse study.

FIG. 23 shows a graph depicting the distributions of temperature differences between tumor treatment groups from an exemplary in vivo mouse study.

FIG. 24 shows a graph depicting temperature differential data over time and as a function of treatment group from tumor-bearing mice in an exemplary in vivo mouse study.

FIG. 25 shows radial smoothing graphs depicting the effect of treatment on temperature differentials in tumor-bearing mice in an exemplary in vivo mouse study.

FIG. 26 shows graphs depicting the temperature change distribution analysis for tumor treatment group by body location from an exemplary in vivo mouse study.

FIG. 27 shows a graph depicting the distributions of temperature differences between tumor treatment groups from an exemplary in vivo mouse study.

FIG. 28 shows a graph depicting temperature differential data over time and as a function of treatment group from tumor-bearing mice in an exemplary in vivo mouse study.

FIG. 29 shows radial smoothing graphs depicting the effect of treatment on temperature differentials in tumor-bearing mice in an exemplary in vivo mouse study.

FIG. 30 shows a schematic depicting exemplary cancer patient monitoring compared to the disclosed method of cancer patient monitoring.

FIG. 31 shows a schematic depicting an exemplary temperature sensor monitor implanted in a human cancer patient.

FIG. 32 shows a schematic depicting exemplary implantation and use of a temperature sensor for monitoring of a human cancer patient's treatment.

FIG. 33 shows a schematic illustrating an exemplary timeline of events corresponding to the disclosed method for determining an oncological status metric.

FIG. 34 shows a schematic illustrating data types associated with an exemplary cancer patient's treatment regimen.

FIG. 35 shows a schematic illustrating sources of information that may be used to determine an oncological status metric for an exemplary cancer patient.

FIG. 36 shows a schematic illustrating data types that may be included in a database of anonymized patient medical records.

FIG. 37 shows a schematic illustrating an exemplary spreadsheet of anonymized patient medical data.

FIG. 38 shows a schematic illustrating exemplary display outputs for an oncological status metric that could be presented to a user of the disclosed method.

FIG. 39 shows a schematic illustrating an exemplary process by which a machine learning model is trained by an existing dataset prior to assessing new data.

FIG. 40 shows a schematic illustrating an exemplary process of training a machine learning model with previous cancer patient data.

### DETAILED DESCRIPTION

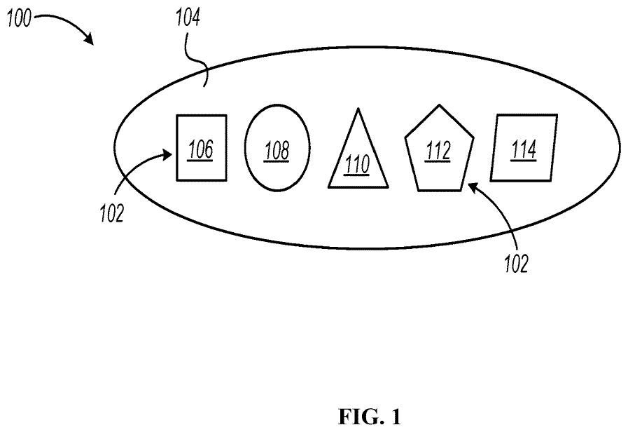

FIG. 1 shows a schematic diagram of an example of a device 100 for measuring one or more physiological characteristics of a subject. The device 100 may include one or more sensors 102. The device 100 may include a device body 104. The device body 104 may include the sensors 102. For example, the device body 104 may partially or fully encapsulate (e.g., contain, encompass, surround, enclose, envelop, or the like) the sensors 102. The device 100 may be implanted into a human or animal subject, for instance implanted into tissue of the subject. The device 100 may be entirely implanted into the human or animal subject. For instance, the device 100 may be entirely contained within the subject. In another example, implantation of the device 100 does not include components outside skin of the subject 200 (e.g., the device may be implanted underneath the skin of the subject 200 so that no portion of the device 100 is outside the skin of the subject 200.

In yet another example, implantation of the device 100 may include the device 100 entirely contained within tissue of an organ of the subject 200. For instance, the device 100 may be sized and shaped for insertion into lung tissue or bladder tissue. In this example, the device 100 may be entirely contained within the lung tissue or the bladder tissue. In another example, the device 100 does not include a pacemaker, defibrillator, or the like. For instance, the device 100 may be substantially smaller than a pacemaker or a defibrillator, for example to facilitate insertion of the device 100 into tissue in correspondence with the tissue entirely containing the device 100.

In a further example, the device 100 may be an oncology monitoring device, for instance a post-operative (or post-implantation) oncology monitoring device. In a still further example, the device 100 may be an implantable fiducial marker. For instance, the device 100 may be imageable in one or more imaging modalities, for example X-ray, CT scan, MRI, or the like. In yet another example, the device 100 may include a contrast agent, for instance to enhance perception of the tumor.

The sensors 102 may measure one or more physiological characteristics of a human or animal subject. In an example, the sensors 102 may include a temperature sensor 106. The temperature sensor 106 may measure temperature of a subject (or temperature of tissue of the subject). In another example, the sensors 102 may include a pH sensor 108. The pH sensor 108 may measure pH of the subject (or pH of tissue of the subject). In yet another example, the sensors 102 may include an oxygen sensor 110. In an example, the oxygen sensor 110 may detect one or more of partial pressure of oxygen (“pO2”), superoxide anions, reactive oxygen species, or the like). The oxygen sensor 110 may measure partial pressure of oxygen of a subject (or partial pressure of oxygen of tissue of the subject). In still yet another example, the sensors 102 may include a glucose level sensor 112. The glucose level sensor 112 may measure glucose level of the subject (or glucose level of tissue of the subject). Accordingly, the sensors 102 may respectively measure one or more physiological characteristics of a subject (or physiological characteristics of tissue of the subject); for example temperature, pH, oxygen, or glucose level.

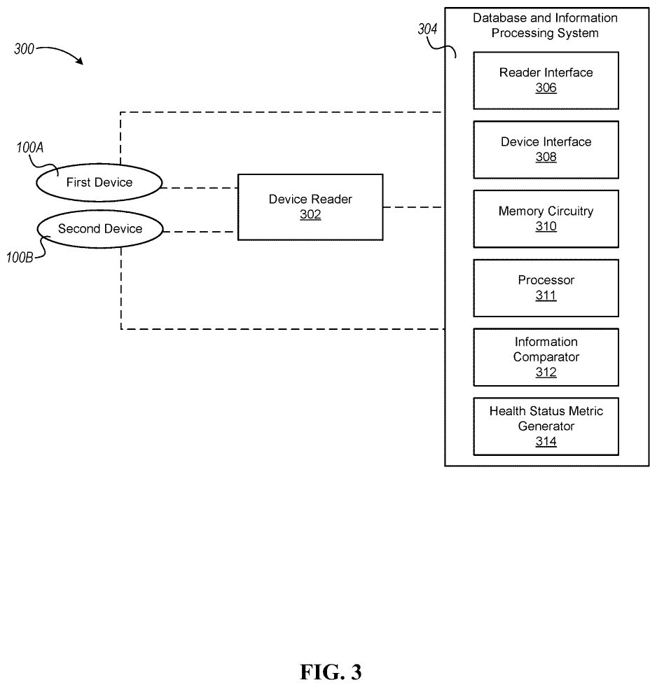

The device 100 may include a device sensor interface 114. The device sensor interface 114 may communicate with the sensors 102. For example, the device sensor interface 114 may receive measured physiological characteristics from the sensors 102. In another example, the device sensor interface 114 may communicate the measured physiological characteristics (provided by the sensors 102). For instance, the device sensor interface 114 may communicate the measured physiological characteristics to allow for storage (or processing) of the measured physiological characteristics. Thus, the device 100 may communicate the measured physiological characteristics to components of a system, for example a physiological characteristic monitoring system (shown in FIG. 3).

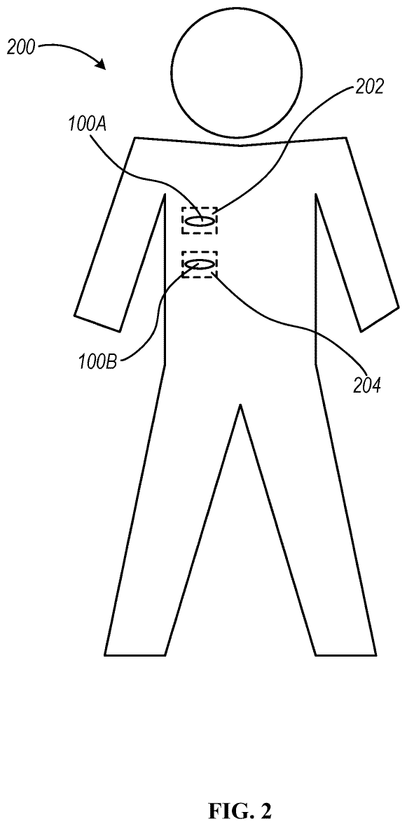

FIG. 2 shows a schematic diagram of an example of a first device 100A and a second device 100B implanted in a subject 200. The subject 200 may include a human or an animal. The first device 100A may include the device 100 (shown in FIG. 1). The second device 100B may include the device 100.

Accordingly, the first device 100A and the second device 100B may measure physiological characteristics of the subject 200. The first device 100A may be implanted at a first location in the subject, for instance implanted at a target location 202 in the subject 200. The first device 100A may measure physiological characteristics of the subject 200 proximate to the first device 100A (or physiological characteristics of tissue of the subject 200 proximate to the first device 100A). For example, the target location 202 may include cancerous tissue, and the first device 100A may measure physiological characteristics of the cancerous tissue. In another example, the target location 202 may include abnormal tissue (e.g., diseased tissue, cancerous tissue, pre-cancerous tissue, or the like), and the first device 100A may measure physiological characteristics of the abnormal tissue.

The second device 100B may be implanted at a second location in the subject 200, for instance implanted at a control location 204 in the subject 200. The second device 100B may measure physiological characteristics of the subject 200 proximate to the second device 100B (or physiological characteristics of tissue of the subject 200 proximate to the second device 100B). For instance, the control location 204 may include non-cancerous tissue, and the second device 100B may measure physiological characteristics of the non-cancerous tissue. In another example, the target location 202 may include healthy (e.g., non-diseased, or the like) tissue, and the second device 100B may measure physiological characteristics of the healthy tissue.

FIG. 3 shows a schematic diagram of a physiological characteristic monitoring system 300 (“system 300”). The system 300 may include one or more of the first device 100A or the second device 100B. The first device 100A (or the second device 100B) may communicate with other components of the system 300, for example to communicate measured physiological characteristics to the other components of the system 300. In another example, the first device 100A (or the second device 100B) may communicate a unique identifier (e.g., a unique serial number, or the like) to other components of the system 300. The unique identifier may be associated with a specific device, for instance the first device 100A or the second device 100B. For example, a first unique identifier may be associated with the first device 100A, and a second unique identifier may be associated with the second device 100B. The first and second unique identifiers may facilitate differentiation between a first set of measured physiological characteristics (measured by the first device 100A) and a second set of measured physiological characteristics (measured by the second device 100B).

The first device 100A may communicate with a device reader 302 of the system 300. The first device 100A may communicate measured physiological characteristics to the device reader 302. For instance, the device reader 302 may interrogate the first device 100A, and the first device 100A may communicate the measured physiological characteristics in response to interrogation by the device reader 302. For example, the first device 100A may be passive (e.g., the first device 100A may lack a battery, or the like), and the device reader 302 may supply energy to the first device 100A to facilitate communication of the measured physiological characteristics to the device reader 302. Passivity of the first device 100A may help minimize the size of the first device 100A and accordingly allow the first device 100A to be implanted in a wide range of locations in the subject 200 (shown in FIG. 2).

The device reader 302 may generate an electromagnetic signal, and the first device 100A may receive the electromagnetic signal. In an example, the device sensor interface 114 (shown in FIG. 1) may receive the electromagnetic signal generated by the device reader 302, and the reception of the electromagnetic signal by the device sensor interface 114 may correspondingly provide power to the sensors 102 (shown in FIG. 1). The device sensor interface 114 may receive measured physiological characteristics from the sensors 102 and communicate the measured physiological characteristics to the device reader 302. In another example, the first device 100A may be active (e.g., the first device 100A may include a battery, or the like), and the first device 100A may communicate with the device reader 302 without the device reader 302 supplying energy to the first device 100A. In an example where the first device 100A is active, the active first device 100A may facilitate different forms of communication between the first device 100A and other components of the system 300, for instance the device reader 302. In an example, the device sensor interface 114 may receive measured physiological characteristics from the sensors 102, and the interface 114 may generate an electromagnetic signal to communicate the measured physiological characteristics to the device reader 302.

The device reader 302 may communicate with a database and information processing system 304. For example, the device reader 302 may communicate measured physiological characteristics (received from one or more of the first device 100A or the second device 100B) to the database and information processing system 304. In an example, the database and information processing system may include a reader interface 306, and the reader interface 306 may communicate with the device reader 302 to receive measured physiological characteristics (that were communicated to the device reader 302 by one or more of the first device 100A or the second device 100B). In another example, the database and information processing system 304 may include a device interface 308. The device interface 308 may facilitate direct communication between the database and information processing system 304 and one or more of the first device 100A and the second device 100B (e.g., the database and information processing system 304 may receive measured physiological characteristics from the first device 100A or the second device 100B; instead of receiving the measured physiological characteristics from the device reader 302). For instance, the device interface 308 may communicate with the device sensor interface 114 (of the device 100, shown in FIG. 1) to facilitate reception of measured physiological characteristics by the database and information processing system 304.

The database and information processing system 304 may include memory circuitry 310. The memory circuitry 310 may store measured physiological characteristics. The system 300 may associate the measured physiological characteristics with a unique identifier associated with the first device 100A (or the second device 100B), and the memory circuitry 310 may store the measured physiological characteristics associated with the unique identifier. For instance, the memory circuitry 310 may store a first set of measured physiological characteristics (measured by the first device 100A) and an associated first unique identifier (corresponding to the first device 100A). The memory circuitry 310 may store a second set of measured physiological characteristics (measured by the second device 100B) and an associated second unique identifier (corresponding to the second device 100B). Accordingly, the database and information processing system 304 may differentiate the first set of measured physiological characteristics from the second set of measured physiological characteristics. In another example, the database and information processing system 304 may include a processor 311, for instance the processor 311 may have processor circuitry. The system 300 may use one or more of the processor 311 or the memory circuitry 310, for instance to record data (e.g., measured physiological characteristics) in the memory circuitry 310. In yet another example, the database information processing system 304 may use one or more of the processor 311 or the memory circuitry 310, for instance to facilitate generation of a health status metric. In another example, the database and information processing system 304 may associate the measured physiological characteristics with a time that the measured physiological characteristics were measured. For instance, a first set of measured physiological characteristics may be measured at a first time, and the memory circuitry 310 may store the first time in association with the first set of measured physiological characteristics. A second set of measured physiological characteristics may be measured at a second time, and the memory circuitry 310 may store the second time in association with the second set of measured physiological characteristics. Accordingly, the database and information processing system 304 may differentiate the first set of measured physiological characteristics from the second set of measured physiological characteristics. Thus, the system 300 may monitor physiological characteristics of a subject over one or more periods of time.

The system 300 may comply with provisions of the U.S. Health Insurance Portability and Accountability Act of 1996 (“HIPAA”). For instance, the system 300 may encrypt one or more of measured physiological characteristics or unique identifiers associated with the subject 200 (shown in FIG. 2). In another example, the system 300 may anonymize the measured physiological characteristics, and only permit authorized users to deanonymize the measured physiological characteristics. In yet another example, the system 300 may only allow access to measured physiological data to authorized users. Accordingly, the system 300 maintains the privacy of the subject 200 (shown in FIG. 2).

The database and information processing system 304 may include an information comparator 312. The information comparator 312 may compare physiological characteristics measured with the first device 100A or the second device 100B. For example, the information comparator 312 may compare a first set of measured physiological characteristics with a second set of measured physiological characteristics. The information comparator 312 may determine a difference between the first set of measured physiological characteristics and the second set of measured physiological characteristics, based on the comparison of the first set of measured physiological characteristics with the second set of measured physiological characteristics.

In an example, the first device 100A may measure a first set of physiological characteristics at a first time (e.g., before treatment of a disease, or the like). The first device 100A may measure a second set of physiological characteristics at a second time (e.g., after one or more courses of treatment of a disease, or the like). The information comparator 312 may compare the first set of physiological characteristics with the second set of physiological characteristics, for instance to determine a difference between the first set of physiological characteristics and the second set of physiological characteristics. Accordingly, the information comparator 312 may determine the difference between measured physiological characteristics between the first time and the second time. In another example, the first device 100A may measure a third set of physiological characteristics at a third time (e.g., after the second time). The information comparator may compare the third set of physiological characteristics with one or more of the first set of physiological characteristics or the second set of physiological characteristics. Thus, the information comparator 312 may determine the difference between measured physiological characteristics between the third time and one or more of the first time and the second time. The information comparator 312 may determine one or more rates of change of measured physiological characteristics. For instance, the information comparator 312 may determine a first rate of change between the first set of physiological characteristics and the second set of physiological characteristics. The information comparator 312 may determine a second rate of change between the third set of physiological characteristics and one or more of the first set of physiological characteristics or the second set of physiological characteristics. As a result, the system 300 monitors physiological characteristics of the subject 200 (shown in FIG. 2), and changes to the physiological characteristics of the subject 200 (e.g., before, during, or after cessation of a course of treatment, or the like).

The database and information processing system 304 may include a health status metric generator 314. The health status metric generator 314 may generate a health status metric, for instance based on differences between measured physiological characteristics. The health status metric may correspond to efficacy of treatment for a disease. For instance, changes in measured physiological characteristics may correspond to a treatment being effective in treating a disease.

In an example, the health status metric may have a value of zero if the differences between measured physiological characteristics are not statistically significant. Statistical significance may be determined using, in one example, a statistical test, for example Welch's t-test, or the like. For instance, the health status metric generator 314 may determine if a difference between the first set of physiological characteristics (measured at the first time) and the second set of physiological characteristics (measured at the second time) is statistically significant. The health status metric generator 314 may determine if a difference between the third set of physiological characteristics (measured at the third time) and one or more of the first or second sets of physiological characteristics is statistically significant. The health status metric may have a value of zero if the differences between measured sets of physiological characteristics are not statistically significant. The health status metric may have a value greater than zero if the differences between measured sets of physiological characteristics are statistically significant. For instance, magnitude of the health status metric may correspond with the degree of statistical significance of differences between measured sets of physiological characteristics. In another example, the magnitude of the health status metric may correspond with magnitude of differences between measured sets of physiological characteristics. For instance, an increase in the difference between measured sets of physiological characteristics may correspondingly increase the magnitude of the health status metric.

In another example, the health status metric may have a value of zero if the differences between measured physiological characteristics refrain from exceeding a difference threshold. For instance, the health status metric may have a value of zero if there is less than ten percent difference between sets of physiological characteristics measured by the system 300. Thus, in this example, the difference threshold corresponds to a difference of less than or equal to ten percent between the sets of physiological characteristics. The health status metric may have a value greater than zero if there is more than ten percent difference between the sets of physiological characteristics. The magnitude of the health status metric may correspond with a magnitude of differences between the sets of physiological characteristics. For instance, the health status metric may have a first value (greater than zero) in correspondence with a fifteen percent difference between the sets of physiological characteristics. The health status metric may have a second value (greater than the first value) in correspondence with a twenty percent difference between the sets of physiological characteristics. Accordingly, the health status metric may change based on changes in the measured physiological characteristics.

As described herein, the health status metric may correspond to efficacy of treatment for a disease. For instance, changes in measured physiological characteristics may correspond to a treatment being effective in treating a disease. In an example, a melanoma treated with adoptive T cell transfer immunotherapy may correspond with a decrease in one or more of body temperature (for instance measured at the control location 204, shown in FIG. 2) or melanoma tissue temperature (for instance measured at the target location 202, shown in FIG. 2). In another example, triple negative breast cancer treated with chemotherapy may correspond with a decline in temperature of the triple negative breast cancer tissue (for instance measured at the target location 202, shown in FIG. 2). In yet another example, colon cancer treated with anti-PD-1 immunotherapy may correspond with a decrease in body temperature (for instance measured at the control location 204, shown in FIG. 2). Accordingly, changes in measured physiological characteristics may correspond to a treatment being effective in treating a disease. Thus, the health status metric (generated by the health status metric generator 314) having a value greater than zero may indicate treatment given for a disease is effective. The changes in physiological characteristics may appear sooner than changes in physical characteristics (e.g., shape, size, color, or the like) of the abnormal tissue. As a result, the system 300 may expedite diagnosis of efficacy of treatment of the abnormal tissue.

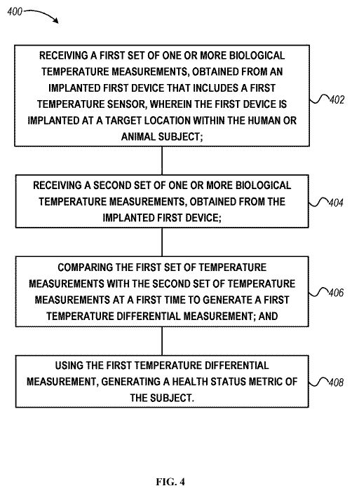

FIG. 4 shows a flowchart of a method 400 of using one or more human-implanted or animal-implanted devices (for instance the first device 100A or the second device 100B, shown in FIGS. 2 and 3) for determining medical diagnostic information related to a course of treatment of a human or animal subject (for instance the subject 200, shown in FIG. 2), for instance a subject having abnormal tissue (e.g., diseased tissue such as cancerous tissue, pre-cancerous tissue, or the like). In describing the method 400, reference is made to one or more components, features, functions and operations previously described herein. Where convenient, reference is made to the components, features, operations, and the like with reference numerals. The reference numerals provided are exemplary and are not exclusive. For instance, components, features, functions, operations, and the like described in the method 400 include, but are not limited to, the corresponding numbered elements provided herein and other corresponding elements described herein (both numbered and unnumbered) as well as their equivalents.

The method 400 may include measuring one or more physiological characteristics, for instance one or more of temperature, pH, oxygen, or glucose level. The measured one or more physiological characteristics may be received, for instance by components of the system 300 (shown in FIG. 3). For example, the method 400 at 402 may include receiving a first set of one or more biological temperature measurements. The first set of one or more biological temperature measurements may be obtained from an implanted first device 100A that includes a first temperature sensor (e.g., temperature sensor 106). The first device 100A may be implanted at a target location 202 within the human or animal subject 200. The target location may include at least a portion of abnormal tissue, for instance cancerous tissue. At 404, the method 400 may include receiving a second set of one or more biological temperature measurements. The second set of one or more biological temperature measurements may be obtained from the implanted first device 100A.

The method 400 may include comparing the measured physiological characteristics, for instance comparing a first set of physiological characteristics with a second set of physiological characteristics. For example, the method 400 may include at 406, comparing the first set of temperature measurements with the second set of temperature measurements. For instance, an information comparator 312 may compare the first set of temperature measurements with the second set of temperature measurements. The comparison of the first set of temperature measurements with the second set of temperature measurements may occur at a first time. In another example, a first temperature differential measurement may be generated based on the comparison of the first set of temperature measurements with the second set of temperature measurements.

The method 400 may include generating a health status metric. In an example, the health status metric may be generated using the measured physiological characteristics. The health status metric may be generated based on comparison of the measured physiological characteristics. The health status metric may be generated based on measurements from two or more of the sensors 102. For example, the health status metric may be generated based on two or more of the temperature measurements, pH measurements, oxygen measurements, or glucose level measurements produced by the sensors 102. For example, at 408, the method 400 may include using the first temperature differential measurement, for instance to generate a health status metric of the subject. In an example, a health status metric generator 314 generates the health status metric.

Several options for the method 400 follow. The method 400 may include administration of a course of treatment to the subject 200. In an example, the course of treatment includes administration of one or more of a first dose of treatment or a second dose of treatment. For example, the first dose of treatment may include a first administration of chemotherapy. The second dose may include a second administration of the chemotherapy. The second dose may include a different form of treatment, for example immunotherapy.

The medical diagnostic information related to a course of treatment may include measured physiological characteristics. For example, the method 400 may include measurement of physiological characteristics prior to initiation of the course of treatment (e.g., before administration of a first dose of treatment, for instance a first dose of chemotherapy). In an example, the physiological characteristics of the subject 200 are measured an hour before, a day before, a week before, or a month before initiation of a course of treatment (or at times therebetween).

The method 400 may include measurement of physiological characteristics during the course of treatment. Stated another way, the physiological characteristics of the subject 200 may be measured after initiation of the course of treatment. In another example, the physiological characteristics of the subject may be measured concurrently with the course of treatment. For example, the physiological characteristics of the subject 200 may be measured after administration of a first dose of treatment, for instance a first dose of immunotherapy. In another example, the physiological characteristics of the subject 200 may be measured after administration of a second dose of treatment, for instance a second dose of immunotherapy (or another form of treatment).

The physiological characteristics of the subject 200 may be measured concurrently with the first dose of treatment, an hour after the first dose of treatment, a day after the first dose of treatment, a month after the first dose of treatment, or the like (or at times therebetween). The physiological characteristics of the subject 200 may be measured concurrently with the second dose of treatment, an hour after the second dose of treatment, a day after the second dose of treatment, a month after the second dose of treatment, or the like. Accordingly, physiological characteristics of the subject may be measured during a course of treatment.

The physiological characteristics may be measured after cessation of the course of treatment. For instance, the physiological characteristics of the subject 200 may be measured after a (planned) final dose of treatment, for example a final dose of chemotherapy. In another example, the physiological characteristics of the subject 200 may be measured after a determination that a disease has been cured, for instance after a determination cancer has entered remission. The physiological characteristics of the subject 200 may be measured an hour after the cessation of the course of treatment, a day after the cessation of the course of treatment, a month after the cessation of the course of treatment, or the like (or at times therebetween). In yet another example, the physiological characteristics of the subject 200 are measured indefinitely after the course of treatment (e.g. months after cessation of the course of treatment, years after the course of treatment, or the like). For instance, the physiological characteristics of the subject are monitored to determine if there is a recurrence in cancer or intensification of disease. Accordingly, the physiological characteristics of the subject may be measured in relation to a course of treatment (e.g., prior to, during, or after cessation of the course of treatment). Thus, a health status metric may be generated in relation to a course of treatment (e.g., prior to, during, or after cessation of the course of treatment). In another example, physiological characteristics of the subject may be measured repeatedly in relation to the course of treatment (e.g., in two or more instances, or the like). The health status metric may be generated repeatedly in relation to a course of treatment (e.g., in two or more instances, or the like).

In another example, the method 400 may include receiving a third set of one or more biological temperature measurements. In an example, the third set of one or more biological temperature measurements may be obtained from a second device I 00B that includes a second temperature sensor (for instance the temperature sensor I 06). The second device I 00B may be implanted at a control location 204 that is different from the target location 202. The method 400 may include comparing the first set of temperature measurements with the third set of temperature measurements at a second time. A second temperature differential measurement may be generated based on the comparison of the first set of temperature measurements with the third set of temperature measurements. The comparison of the first set of temperature measurements with the third set of temperature measurements may occur at a second time. In another example, the method 400 may include generating the health status metric of the subject 200, for instance using the first and second temperature differential measurements.

In yet another example, the method 400 may include identifying a difference between the first and second temperature differential measurements. The difference between the first and second temperature differential measurements may be used to generate the health status metric of the subject 200. In still yet another example, the method 400 may include treating the subject 200. The treatment may include one or more of immunotherapy, chemotherapy, or radiation therapy.

In a further example, the method 400 may include implanting the first device 100A at the target location 202 within the subject 200. In a still further example, the method 400 may include implanting the second device 100B at the control location 204 within the subject 200. In a still yet further example, the method 400 may include receiving one or more of a first pH value, a first oxygen value, or a first glucose value. One or more of a second pH value, a second oxygen value, or a second glucose value may be received, for instance by a device reader 302. The first pH value, second pH value, first oxygen value, second oxygen value, first glucose value, or the second glucose value may be obtained from one or more of a pH sensor 108, an oxygen sensor 110, or a glucose level sensor 112.

The method 400 may include comparing the first pH value with the second pH value, for instance to generate a first pH differential measurement. In an example, the information comparator 312 may compare the first pH value with the second pH value. The information comparator 312 may generate the first pH differential measurement. The health status metric of the subject 200 may be generated using the first pH differential measurement. The method 400 may include comparing the first oxygen value with the second oxygen value, for instance to generate a first oxygen differential measurement. In an example, the information comparator 312 may compare the first oxygen value with the second oxygen value. The information comparator 312 may generate the first oxygen differential measurement. The health status metric of the subject 200 may be generated using the first oxygen differential measurement. The method 400 may include comparing the first glucose value with the second glucose value, for instance to generate a first glucose differential measurement. In an example, the information comparator 312 may compare the first glucose value with the second glucose value. The information comparator 312 may generate the first glucose differential measurement. The health status metric of the subject 200 may be generated using the first glucose differential measurement.

The method 400 may include that the health status metric includes an indication of whether the course of treatment is effective in treating the abnormal tissue, for instance cancerous tissue, of the subject 200. For instance, the indication may be a value of zero corresponding to the treatment being ineffective in treating the cancerous tissue of the subject 200. The indication may be a value greater than zero corresponding to the treatment being effective in treating the cancerous tissue of the subject 200.

The course of treatment may be continued based on the indication of whether the course of treatment is effective in treating the abnormal tissue of the subject 200. For instance, the course of treatment may be continued if the treatment is effective. In another example, additional sets of measured physiological characteristics are measured after continuing the course of treatment of the subject 200. For example, a first dose of treatment in the course of treatment is administered to the subject 200. In some examples, the first dose of treatment in the course of treatment may be determined to be effective, for instance using the health status metric. In another example, the first dose of treatment may be determined to be ineffective, for instance using the health status metric. A second dose in the course of treatment may be administered to the subject 200. The second dose may be indicated to be effective, for instance by measuring one or more physiological characteristics after the second dose is administered, and generating the health status metric based on the physiological characteristics measured after the second dose is administered. Accordingly, efficacy of the course of treatment is monitored, for instance using one or more components of the system 300.

The course of treatment may be discontinued based on the indication of whether the course of treatment is effective in treating the abnormal tissue of the subject 200. For instance, the course of treatment may be discontinued if the course of treatment is determined to be ineffective, for instance using the health status metric. In yet another example, the course of treatment may be changed based on the indication of whether the course of treatment is effective in treating the abnormal tissue of the subject. For instance, a first dose in the course of treatment may be administered to the subject 200. An amount (e.g., volume, concentration, duration, or the like) of treatment may be changed based on the indication of whether the course of treatment is effective in treating the abnormal tissue of the subject 200 (e.g., by increasing the amount of treatment given to the patient, for instance by doubling the amount of the first dose). The course of treatment may be changed to provide a different treatment. For instance, the first dose in the course of treatment may use chemotherapy. A second dose in the course of treatment may be administered to the patient. The second dose may include immunotherapy. Accordingly, the treatment is changed during the course of treatment, for instance if the first dose is determined to be ineffective or the first dose is determined to be effective and the second dose enhances effectiveness of the first dose.

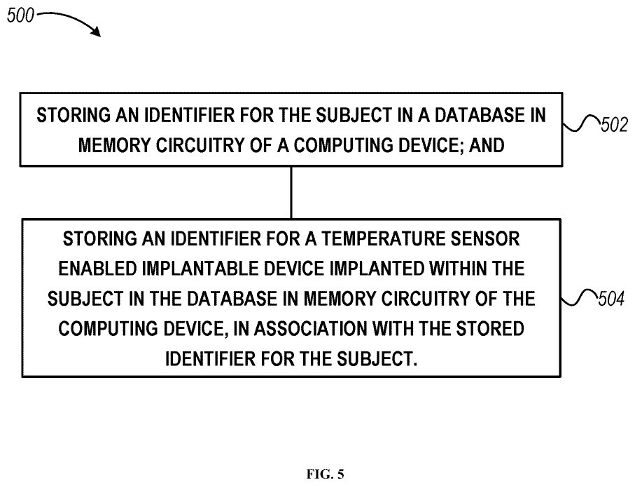

FIG. 5 shows a flowchart of an example of a computer-implemented method 500 for monitoring a subject of a medical treatment. In describing the method 500, reference is made to one or more components, features, functions and operations previously described herein. Where convenient, reference is made to the components, features, operations, and the like with reference numerals. The reference numerals provided are exemplary and are not exclusive. For instance, components, features, functions, operations, and the like described in the method 500 include, but are not limited to, the corresponding numbered elements provided herein and other corresponding elements described herein (both numbered and unnumbered) as well as their equivalents.

The method 500 may include at 502, storing an identifier for the subject 200 in a database in memory circuitry 310 of a computing device (e.g., the database and information processing system 304, shown in FIG. 3). At 504, the method 500 may include storing an identifier for a temperature sensor (e.g., temperature sensor 106, shown in FIG. 1) enabled implantable device 100 implanted within the subject 200 in the database in memory circuitry 310 of the computing device. The identifier for the temperature sensor may be stored in association with the stored identifier for the subject 200.

Several options for the method 500 follow. For example, the method 500 may include storing, in the database, a first set of temperature measurements obtained from the implantable device 100. In another example, the method 500 may include storing, in the database, treatment information related to the subject 200. In yet another example, the method 500 may include storing, in the database, information related to implantation of the device 100 within the subject 200, for example one or more of location or orientation of the device 100 implanted within the subject 200. In still yet another example, the method 500 may include storing, in the database, information related to the subject 200, for example one or more of abnormal body tissue (e.g., diseased tissue, cancerous tissue, pre-cancerous tissue, or the like) or pathologies present with the subject 200. One or more of the first set of temperature measurements, the identifier for the subject 200, the identifier for the temperature sensor, the treatment information related to the subject 200, the information related to implantation of the device 100, or information related to one or more of abnormal body tissue or pathologies present with the subject 200 may be stored in a manner to comply with HIPAA.

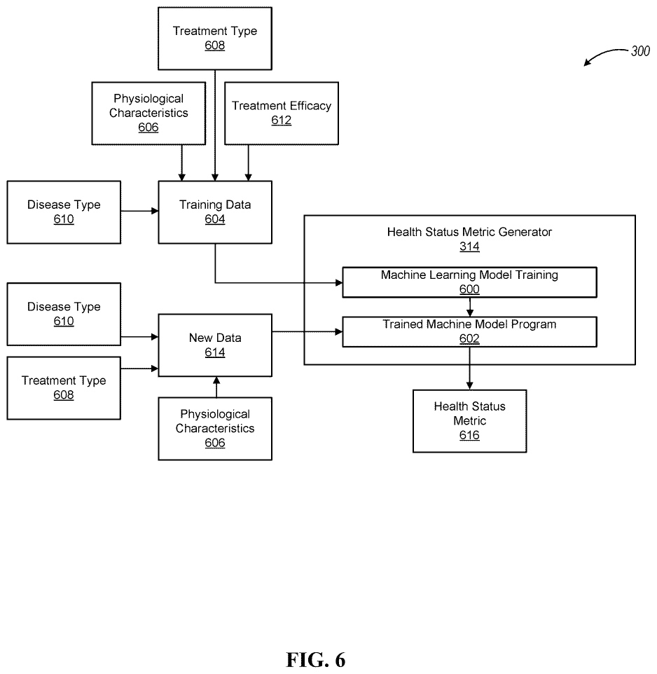

FIG. 6 shows a schematic diagram of another example of the system 300. The system 300 may include the health status metric generator 314. The health status metric generator 314 may include a machine learning algorithm. The machine learning algorithm may include one or more of a supervised, semi-supervised, unsupervised, or reinforcement algorithm. For example, the machine learning algorithm may include a classifier, for instance an Artificial Neural Network, Support Vector Machine, K-Nearest Neighbor, or the like. The health status metric generator 314 may use the machine learning algorithm to determine the health status metric.

In an example, the health status metric generator 314 may include machine learning model training 600. The machine learning model training 600 may generate a trained machine learning model 602. For instance, the machine learning model training 600 may receive training data 604. The reception of the training data 604 by the machine learning model training 600 may generate the trained machine learning model 602.

In an example, the training data 604 includes one or more physiological characteristics 606. For instance, the sensors 102 (shown in FIG. 1) may measure the one or more physiological characteristics 606. The training data 604 may include the measured one or more physiological characteristics 606. In an example, the physiological characteristics 606 include one or more of temperature, pH, oxygen, or glucose value (or combinations thereof, or the like).

The training data 604 may include treatment type 608. For example, a user may input the treatment type 608 into the training data 604. The treatment type 608 may be associated with the measured physiological characteristics 606. For instance, a first set of one or more physiological characteristics may be associated with a first course of treatment. A second set of one or more physiological characteristics may be associated with a second course of treatment.

The training data 604 may include disease type 610, and the disease type 610 may include one or more diseases. For example, a user may input the disease type 610 into the training data 604. In an example, the disease type 610 may include one or more types of cancer. The disease type 610 may include metastasized cancer. The disease type 610 may include diseases other than cancer, for instance an infection. In some examples, the disease type 610 does not include infection. In another example, the disease type 610 may include an autoimmune disease. In yet another example, the disease type 610 may be associated with one or more of the treatment type 608 or the measured physiological characteristics 606. For instance, a first set of one or more physiological characteristics may be associated with a first course of treatment and a first disease type. A second set of one or more physiological characteristics may be associated with a second course of treatment and the first disease type (or a second disease type). Accordingly, the physiological characteristics 606 may be correlated with one or more of the treatment type 608 or the disease type 610.

The training data 604 may include treatment efficacy 612. For example, a user may input the treatment efficacy 612 into the training data 604. The treatment efficacy 612 may correspond with efficacy of a course of treatment in treating a disease. In an example, the treatment efficacy 612 may be positive and correspond with a complete remission of cancer in a subject, for instance the subject 200 (shown in FIG. 2). For example, the treatment efficacy 612 may be positive and correspond with elimination of tumors detected with medical imaging or other medical tests, such as a blood test. In another example, the treatment efficacy 612 may be positive and correspond with a partial remission of the cancer. For instance, the treatment efficacy 612 may be positive and correspond with partial elimination of tumors detected with imaging or other tests. In another example, the treatment efficacy 612 may be positive and correspond with a decrease in size of cancerous tissue (e.g., a decrease in size of a tumor, or the like). In yet another example, the treatment efficacy 612 may be positive and correspond with maintaining size of cancerous tissue (e.g., a tumor does not increase in size or decrease in size, or the like). In still yet another example, the treatment efficacy 612 may be negative and correspond with maintaining size of cancerous tissue. In a further example, the treatment efficacy 612 may be negative and correspond with an increase in size of cancerous tissue (or an increase in cancer markers in a blood test, or the like). Accordingly, the treatment efficacy may be positive or negative, based on changes (or lack of changes) in physical size of cancerous tissue.

In another example, the treatment efficacy 612 may be associated with one or more of the treatment type 608, the measured physiological characteristics 606, or the disease type 610. For instance, a first set of one or more physiological characteristics may be associated with a first course of treatment, a first disease type, and a first treatment efficacy. A second set of one or more physiological characteristics may be associated with a second course of treatment, the first disease type (or a second disease type), and a second treatment efficacy. Accordingly, the physiological characteristics 606 may be correlated with one or more of the treatment type 608, the disease type 610, or the treatment efficacy 612.

As described herein, the reception of the training data 604 by the machine learning model training 600 may generate the trained machine learning model 602. The trained machine learning model 602 may use new data 614, for instance to generate health status metric 616. For example, the trained machine learning model 602 may use the new data 614 in combination with a (trained) machine learning algorithm to generate the health status metric 616. For instance, the health status metric 616 may correspond with efficacy of treatment (e.g., positive, negative, a value of zero, a value greater than zero, or the like).

In an example, the new data 614 may include one or more physiological characteristics 606, for instance physiological characteristics 606 measured from the subject 200 (shown in FIG. 2). In contrast, the training data 604 may use the physiological characteristics 606 of a plurality of training subjects (in some examples excluding the subject 200). In another example, the new data 614 may include treatment type 608, for instance one or more doses of chemotherapy administered to the subject 200. In contrast, the training data 604 may use the treatment type 608 of a plurality of training subjects (in some examples excluding the subject 200). In yet another example, the new data 614 may include disease type 610, for instance a type of cancer afflicting the subject 200. In contrast, the training data 604 may use the disease type 610 of a plurality of training subjects (in some examples excluding the subject 200). Accordingly, the health status metric 616 may be based on one or more of the physiological characteristics 606 measured with respect to the subject 200, the treatment type 608 associated with the subject 200, or the disease type 610 associated with the subject.

The health status metric 616 may correspond with efficacy of treatment (e.g., positive, negative, a value of zero, a value greater than zero, or the like). The health status metric 616 may facilitate differentiation between an infection response and a response of cancer to treatment. For instance, the training data 604 may include one or more of physiological characteristics 606 corresponding with presence of an infection in a subject (or plurality of subjects). The training data 604 may include one or more physiological characteristics 606 corresponding with presence of cancer in a subject (or a plurality of subjects).

The training data 604 may include one or more physiological characteristics corresponding with presence of an autoimmune disease in a subject (or a plurality of subjects). The training data 604 may include one or more physiological characteristics corresponding with a healthy (non-diseased) subject (or plurality of subjects). Accordingly, the trained machine learning model 602 may use the training data 604 to differentiate between a healthy subject, a subject afflicted with cancer, a subject experiencing infection, or a subject afflicted with an autoimmune disease. In another example, the system 300 facilitates monitoring of efficacy of a course of treatment administered to the subject 200. As a result, treatment of disease is enhanced, for instance because the device 100 (or the system 300) may facilitate determining whether a particular course of treatment is effective in treatment of the disease (or whether a response is related to infection, autoimmune disease, or the like).

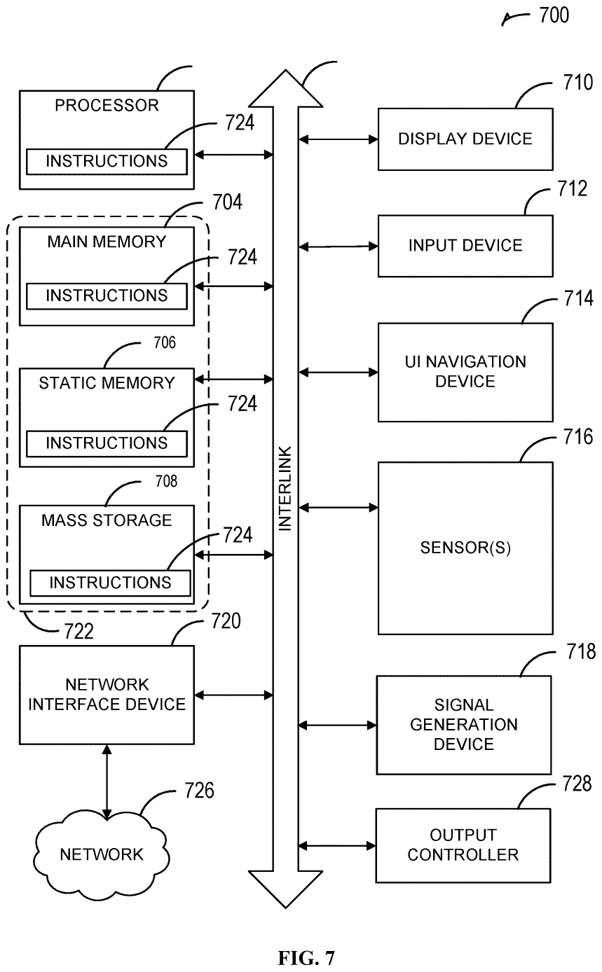

FIG. 7 shows a block diagram of an example machine 700 upon which any one or more of the techniques (e.g., methodologies, methods, operations, or the like) discussed herein may perform. Examples, as described herein, may include, or may operate by, logic or a number of components, or mechanisms in the machine 700. Circuitry (e.g., processing circuitry) is a collection of circuits implemented in tangible entities of the machine 700 that include hardware (e.g., simple circuits, gates, logic, etc.). Circuitry membership may be flexible over time. Circuitries include members that may, alone or in combination, perform specified operations when operating. In an example, hardware of the circuitry may be immutably designed to carry out a specific operation (e.g., hardwired).

In an example, the hardware of the circuitry may include variably connected physical components (e.g., execution units, transistors, simple circuits, etc.) including a machine readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuitry in hardware via the variable connections to carry out portions of the specific operation when in operation. Accordingly, in an example, the machine readable medium elements are part of the circuitry or are communicatively coupled to the other components of the circuitry when the device is operating. In an example, any of the physical components may be used in more than one member of more than one circuitry. For example, under operation, execution units may be used in a first circuit of a first circuitry at one point in time and reused by a second circuit in the first circuitry, or by a third circuit in a second circuitry at a different time. Additional examples of these components with respect to the machine 700 follow.

In alternative embodiments, the machine 700 may operate as a standalone device or may be connected (e.g., networked) to other machines. In a networked deployment, the machine 700 may operate in the capacity of a server machine, a client machine, or both in server-client network environments. In an example, the machine 700 may act as a peer machine in peer-to-peer (P2P) (or other distributed) network environment. The machine 700 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

The machine (e.g., computer system) 700 may include a hardware processor 702 (e.g., a central processing unit (CPU), a graphics processing unit (GPU), a hardware processor core, or any combination thereof), a main memory 704, a static memory (e.g., memory or storage for firmware, microcode, a basic-input-output (BIOS), unified extensible firmware interface (UEFI), etc.) 706, and mass storage 708 (e.g., hard drive, tape drive, flash storage, or other block devices) some or all of which may communicate with each other via an interlink (e.g., bus) 730. The machine 700 may further include a display unit 710, an alphanumeric input device 712 (e.g., a keyboard), and a user interface (UI) navigation device 714 (e.g., a mouse). In an example, the display unit 710, input device 712 and UI navigation device 714 may be a touch screen display. The machine 700 may additionally include a storage device (e.g., drive unit) 708, a signal generation device 718 (e.g., a speaker), a network interface device 720, and one or more sensors 716, such as a global positioning system (GPS) sensor, compass, accelerometer, or other sensor. The machine 700 may include an output controller 728, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

Registers of the processor 702, the main memory 704, the static memory 706, or the mass storage 708 may be, or include, a machine readable medium 722 on which is stored one or more sets of data structures or instructions 724 (e.g., software) embodying or utilized by any one or more of the techniques or functions described herein. The instructions 724 may also reside, completely or at least partially, within any of registers of the processor 702, the main memory 704, the static memory 706, or the mass storage 708 during execution thereof by the machine 700. In an example, one or any combination of the hardware processor 702, the main memory 704, the static memory 706, or the mass storage 708 may constitute the machine readable media 722. While the machine readable medium 722 is illustrated as a single medium, the term “machine readable medium” may include a single medium or multiple media (e.g., a centralized or distributed database, and/or associated caches and servers) configured to store the one or more instructions 724.

The term “machine readable medium” may include any medium that is capable of storing, encoding, or carrying instructions for execution by the machine 700 and that cause the machine 700 to perform any one or more of the techniques of the present disclosure, or that is capable of storing, encoding or carrying data structures used by or associated with such instructions. Non-limiting machine readable medium examples may include solid-state memories, optical media, magnetic media, and signals (e.g., radio frequency signals, other photon based signals, sound signals, etc.). In an example, a non-transitory machine readable medium comprises a machine readable medium with a plurality of particles having invariant (e.g., rest) mass, and thus are compositions of matter. Accordingly, non-transitory machine-readable media are machine readable media that do not include transitory propagating signals. Specific examples of non-transitory machine readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 724 may be further transmitted or received over a communications network 726 using a transmission medium via the network interface device 720 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.11.4 family of standards, peer-to-peer (P2P) networks, among others. In an example, the network interface device 720 may include one or more physical jacks (e.g., Ethernet, coaxial, or phone jacks) or one or more antennas to connect to the communications network 726. In an example, the network interface device 720 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 700, and includes digital or analog communications signals or other intangible medium to facilitate communication of such software. A transmission medium is a machine readable medium.

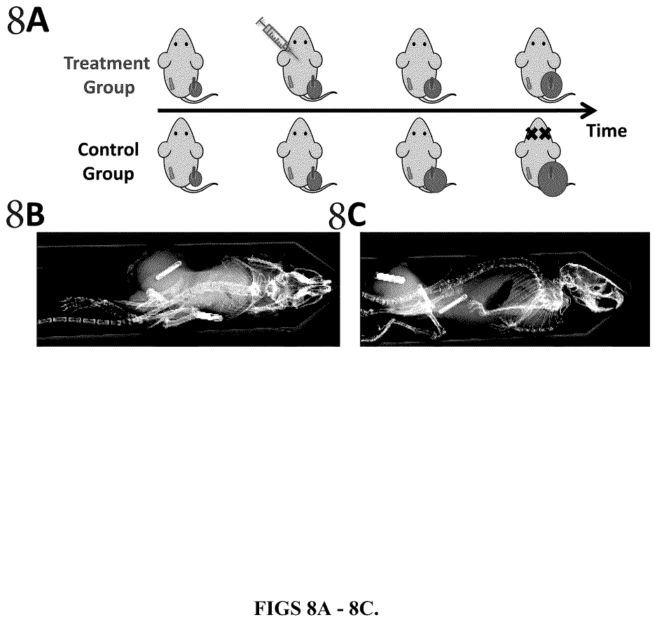

FIG. 8A is a schematic of an exemplary study design and temperature chip placement verified by X-ray. For each cancer model and its corresponding therapy, mice were divided into the treatment group and the control group. Both groups followed the same recording protocol. Note that two chips were implanted on both tumor-bearing mice: one on the left flank the other one the right flank inside the tumor. Magnification X-rays were acquired with Faxitron Specimen Radiography System (Hologic, Santa Clara, CA).

FIG. 8B is a post-mortem X-ray image of a B16 tumor on a C57BL/6J mouse. Note that two chips were implanted on tumor-bearing mice: one on the left flank the other one the right flank inside the tumor. Magnification X-rays were acquired with Faxitron Specimen Radiography System (Hologic, Santa Clara, CA).

FIG. 8C is a post-mortem X-ray image of a 4T1 tumor on a BALB/c mouse. Note that two chips were implanted on tumor-bearing mice: one on the left flank the other one the right flank inside the tumor. Magnification X-rays were acquired with Faxitron Specimen Radiography System (Hologic, Santa Clara, CA).

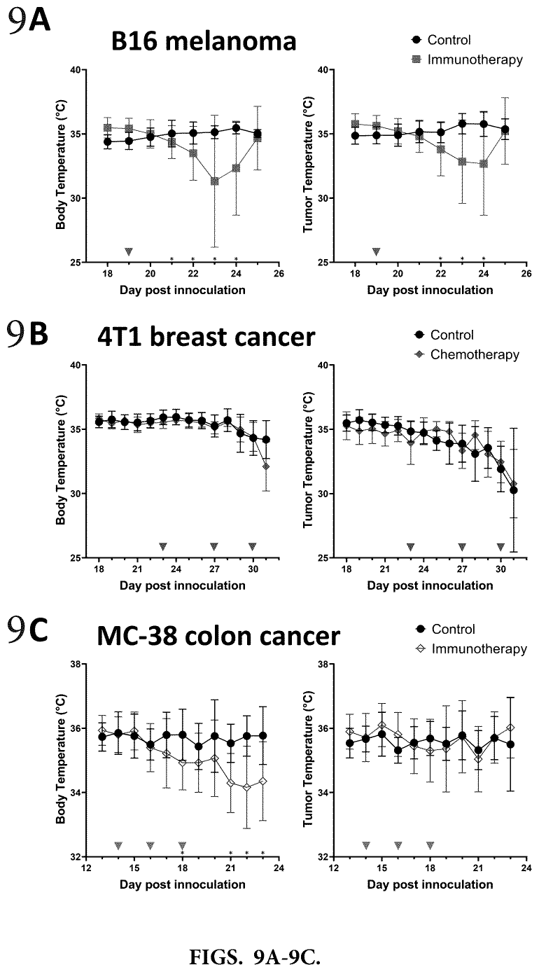

FIG. 9A is a temperature vs. time graph that shows data from an exemplary study of the temperature of mouse bodies and tumors, in an exemplary cancer model, responding to an corresponding exemplary cancer treatment. B16 melanoma-bearing mice (control, n=10) and its response to TRP-2 T cells adoptive transfer (immunotherapy, n=8). Data are presented with average (symbol)±standard deviation (error bar). Left, body temperature; right, tumor temperature. Days when therapy was administered are labeled with ▾. Days with statistically significant difference (P<0.05) between the control and the treatment group is denoted with *.

FIG. 9B is a temperature vs. time graph that shows data from an exemplary study of the temperature of mouse bodies and tumors, in an exemplary cancer model, responding to an corresponding exemplary cancer treatment. 4T1 TNBC-bearing mice (control, n=10) and its response to AC-T chemotherapy (n=6). Data are presented with average (symbol)±standard deviation (error bar). Left, body temperature; right, tumor temperature. Days when therapy was administered are labeled with ▾. Days with statistically significant difference (P<0.05) between the control and the treatment group is denoted with *.

FIG. 9C is a temperature vs. time graph that shows data from an exemplary study of the temperature of mouse bodies and tumors, in an exemplary cancer model, responding to an corresponding exemplary cancer treatment. MC-38 colon cancer-bearing mice (control, n=11) and its response of receiving anti-PD-1 antibody (immunotherapy, n=7). Data are presented with average (symbol)±standard deviation (error bar). Left, body temperature; right, tumor temperature. Days when therapy was administered are labeled with ▾. Days with statistically significant difference (P<0.05) between the control and the treatment group is denoted with *.

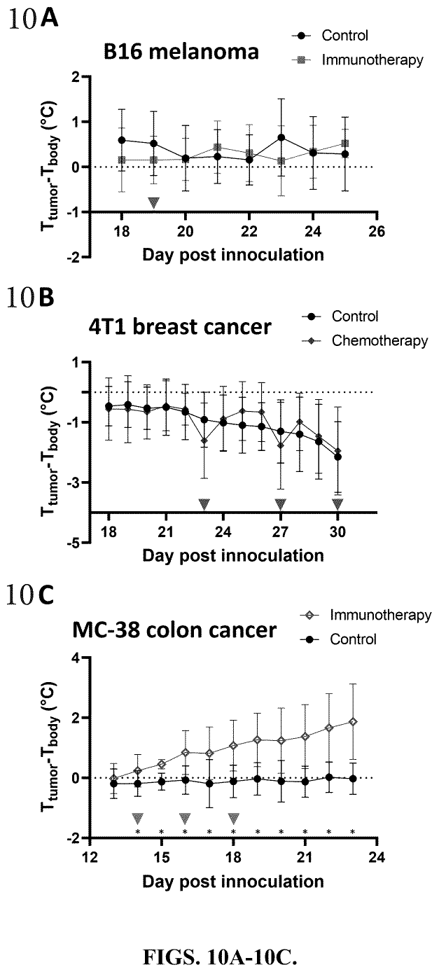

FIG. 10A is a temperature vs. time graph that shows data from an exemplary study on temperature differences (ΔT, tumor temperature minus body temperature) between mouse tumors and mouse bodies, and their response to an exemplary melanoma treatment: B16 melanoma adaptive T cell transfer immunotherapy. Data are presented with average (symbol)=standard deviation (error bar). Days when therapy was administered are labeled with ▾. Days with statistically significant difference (P<0.05) between the control and the treatment group is denoted with *.

FIG. 10B is a temperature vs. time graph that shows data from an exemplary study on temperature differences (ΔT, tumor temperature minus body temperature) between mouse tumors and mouse bodies, and their response to an exemplary breast cancer treatment: 4T1 TNBC AC-T chemotherapy. Data are presented with average (symbol)±standard deviation (error bar). Days when therapy was administered are labeled with ▾. Days with statistically significant difference (P<0.05) between the control and the treatment group is denoted with *.

FIG. 10C is a temperature vs. time graph that shows data from an exemplary study on temperature differences (ΔT, tumor temperature minus body temperature) between mouse tumors and mouse bodies, and their response to an exemplary colon cancer treatment: MC-38 colon cancer anti-PD-1 immunotherapy. Data are presented with average (symbol)±standard deviation (error bar). Days when therapy was administered are labeled with ▾. Days with statistically significant difference (P<0.05) between the control and the treatment group is denoted with *.

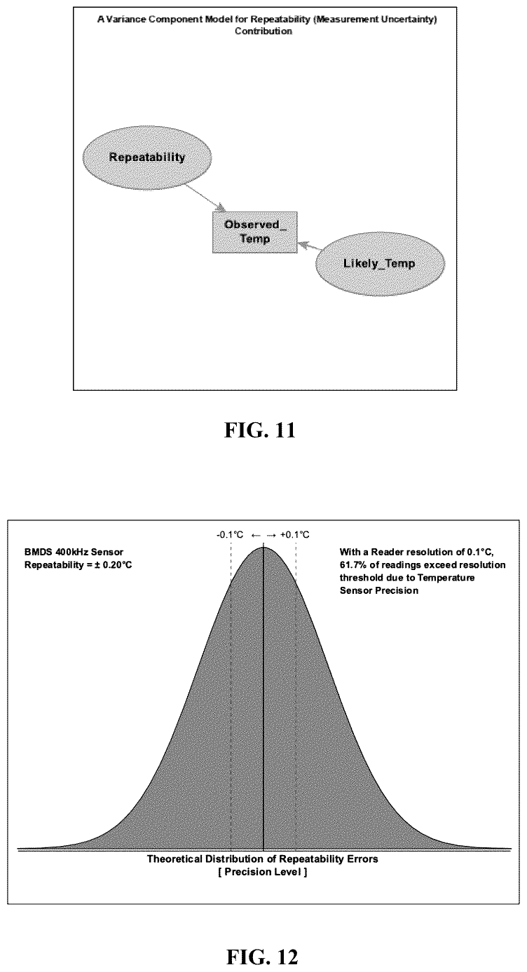

FIG. 11 is a schematic depicting an SEM path analysis, wherein every observed temperature value results from a likely temperature value.

FIG. 12 is a schematic depicting the theoretical repeatability impact distribution of a 400 kHz device used in an exemplary in vivo mouse study.

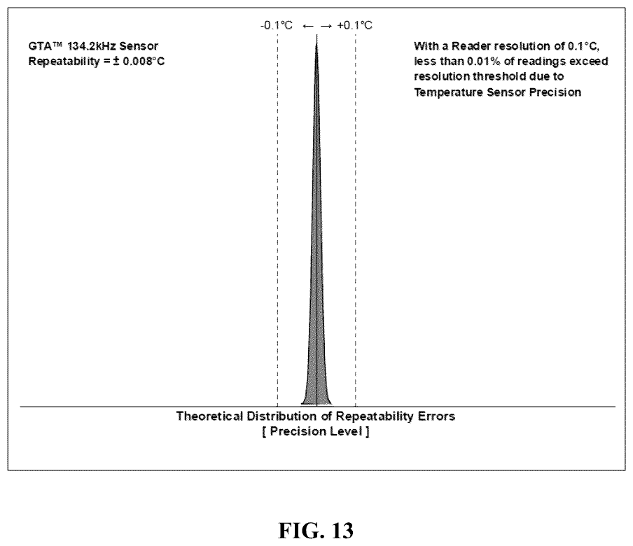

FIG. 13 is a schematic depicting the theoretical repeatability impact distribution of a 134.2 kHz device used in an exemplary in vivo mouse study.

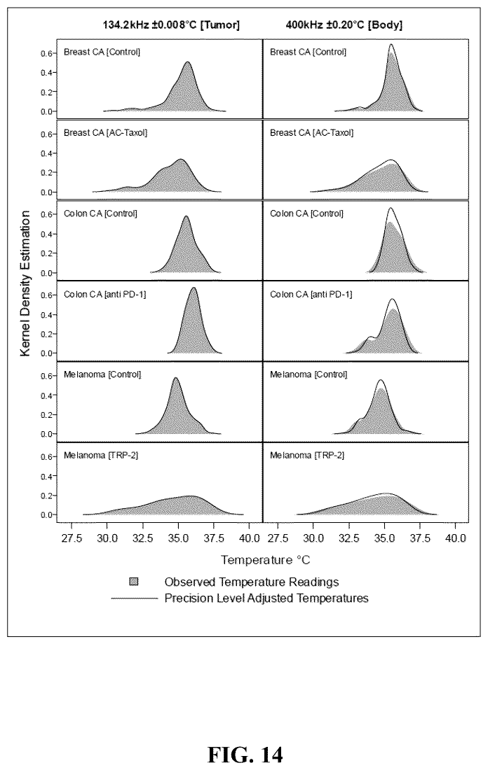

FIG. 14 shows graphs depicting the device uncertainty corrections using a kernel density function, for the observed temperatures from each treatment arm in an exemplary in vivo mouse study.

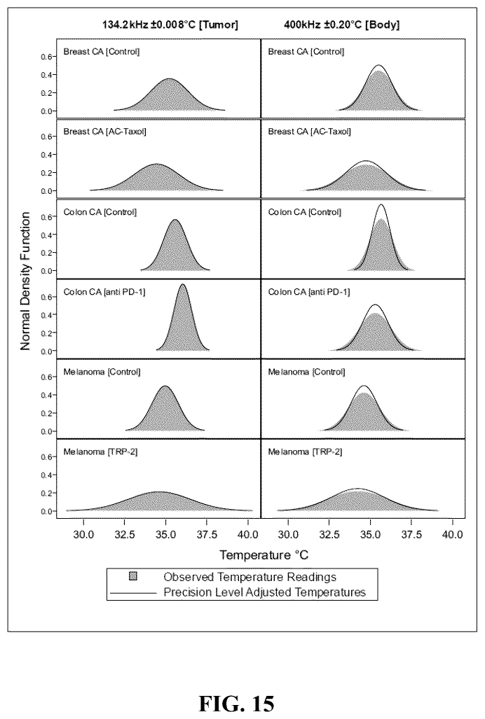

FIG. 15 shows graphs depicting the device uncertainty corrections using a normal density function, for the observed temperatures from each treatment arm in an exemplary in vivo mouse study.

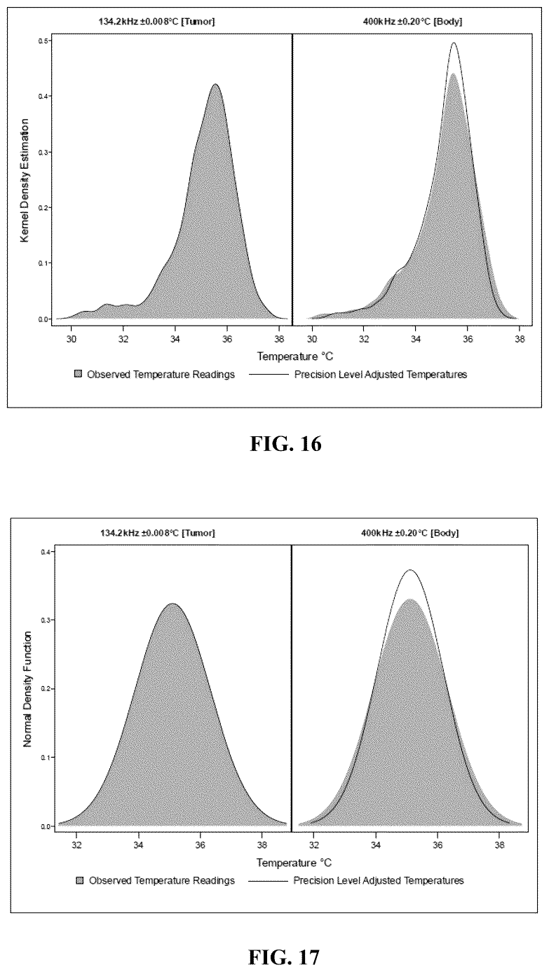

FIG. 16 shows graphs depicting the device uncertainty corrections using a kernel density function for observed temperatures from the combined treatment arms of an exemplary in vivo mouse study.

FIG. 17 shows graphs depicting the device uncertainty corrections using a normal density function for observed temperatures from the combined treatment arms of an exemplary in vivo mouse study.

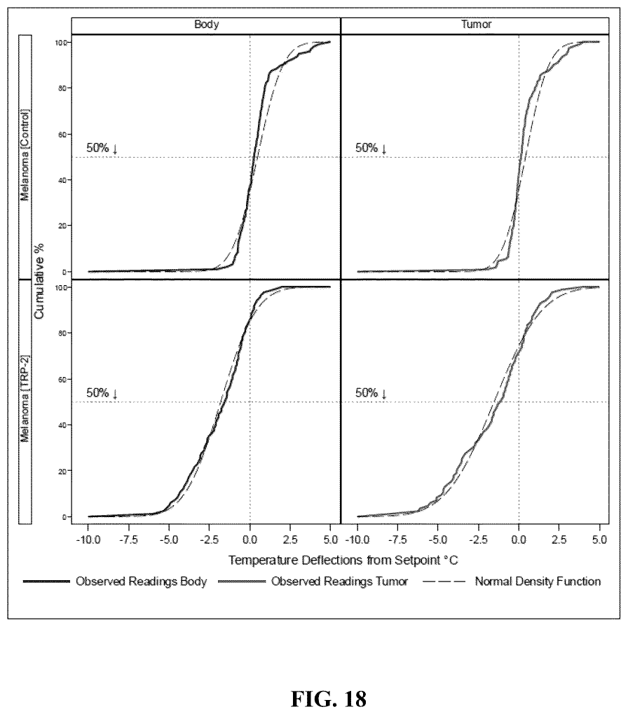

FIG. 18 shows graphs depicting the temperature change distribution analysis for the melanoma treatment groups by body location from an exemplary in vivo mouse study.

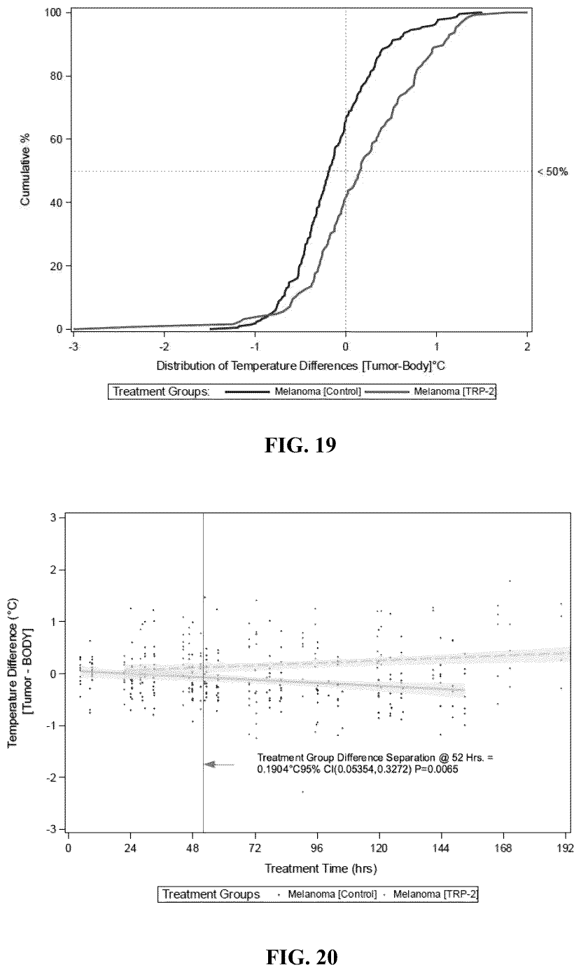

FIG. 19 shows a graph depicting the distributions of temperature differences between melanoma treatment groups from an exemplary in vivo mouse study.

FIG. 20 shows a graph depicting temperature differential data over time and as a function of treatment group from melanoma-afflicted mice in an exemplary in vivo mouse study.

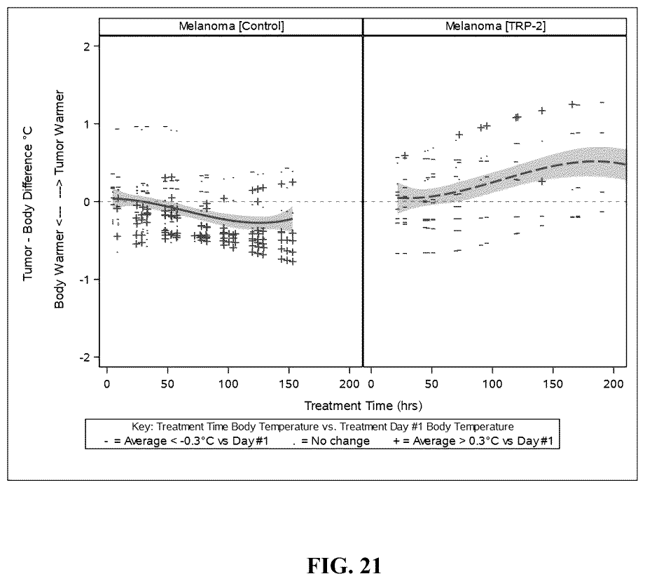

FIG. 21 shows radial smoothing graphs depicting the effect of treatment on temperature differentials in melanoma-afflicted mice in an exemplary in vivo mouse study.

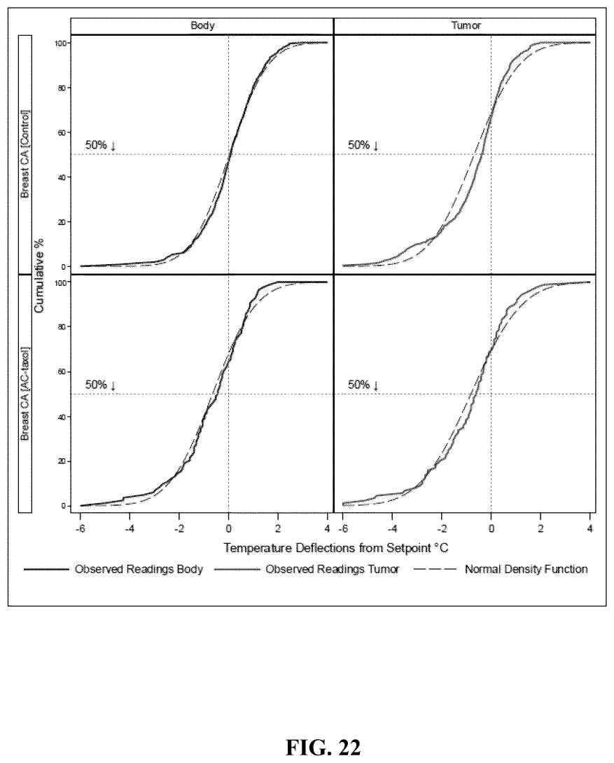

FIG. 22 shows graphs depicting the temperature change distribution analysis for the breast cancer treatment groups by body location from an exemplary in vivo mouse study.

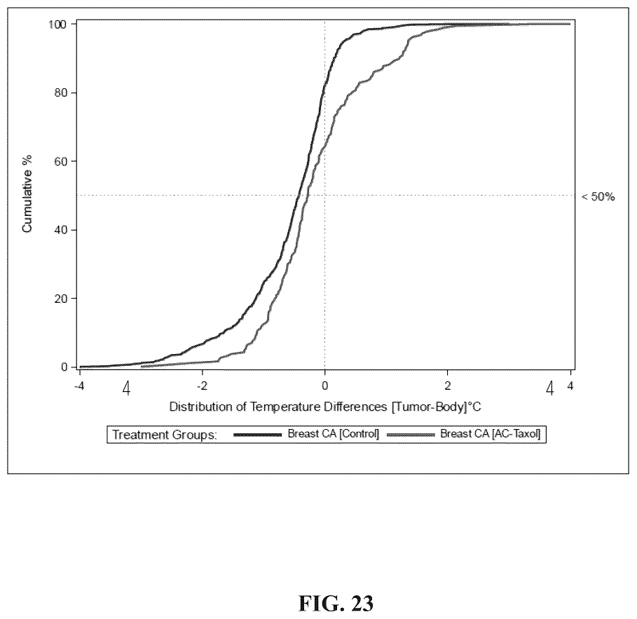

FIG. 23 shows a graph depicting the distributions of temperature differences between breast cancer treatment groups from an exemplary in vivo mouse study.

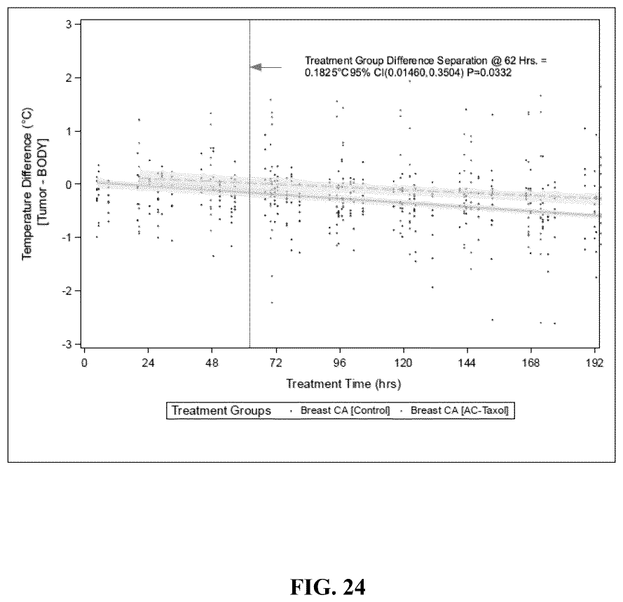

FIG. 24 shows a graph depicting temperature differential data over time and as a function of treatment group from breast cancer-afflicted mice in an exemplary in vivo mouse study.

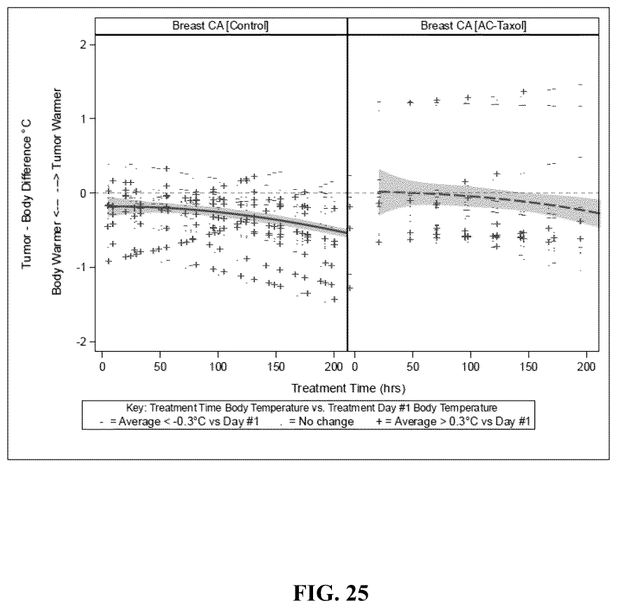

FIG. 25 shows radial smoothing graphs depicting the effect of treatment on temperature differentials in breast cancer-afflicted mice in an exemplary in vivo mouse study.

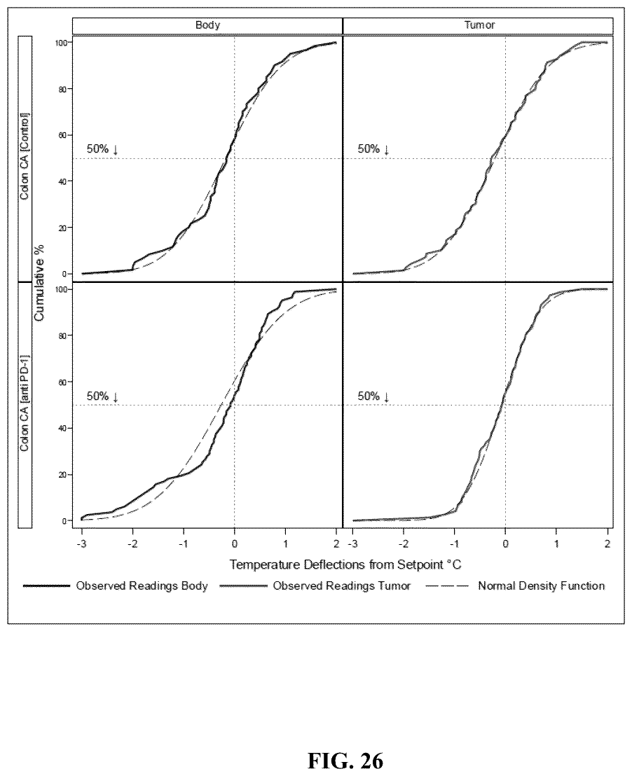

FIG. 26 shows graphs depicting the temperature change distribution analysis for the colon cancer treatment groups by body location from an exemplary in vivo mouse study.

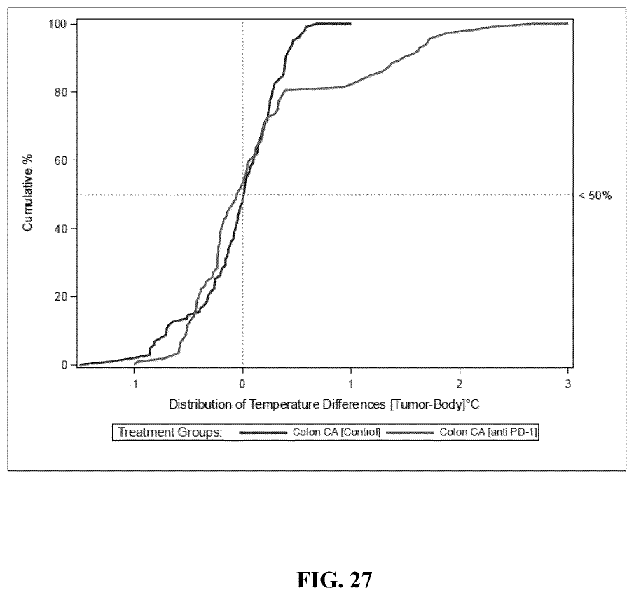

FIG. 27 shows a graph depicting the distributions of temperature differences between colon cancer treatment groups from an exemplary in vivo mouse study.

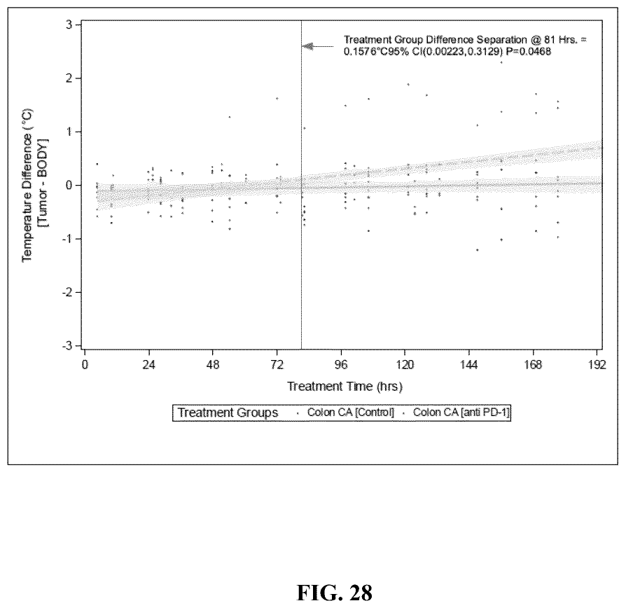

FIG. 28 shows a graph depicting temperature differential data over time and as a function of treatment group from colon cancer-afflicted mice in an exemplary in vivo mouse study.

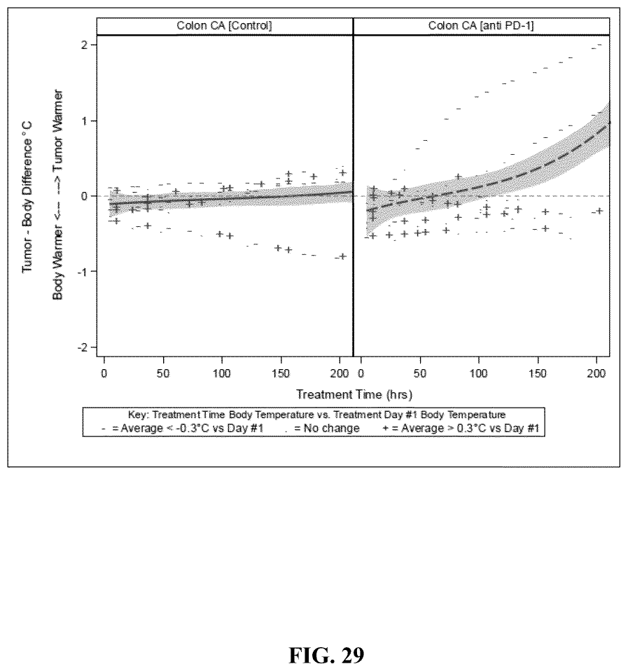

FIG. 29 shows radial smoothing graphs depicting the effect of treatment on temperature differentials in colon cancer-afflicted mice in an exemplary in vivo mouse study.

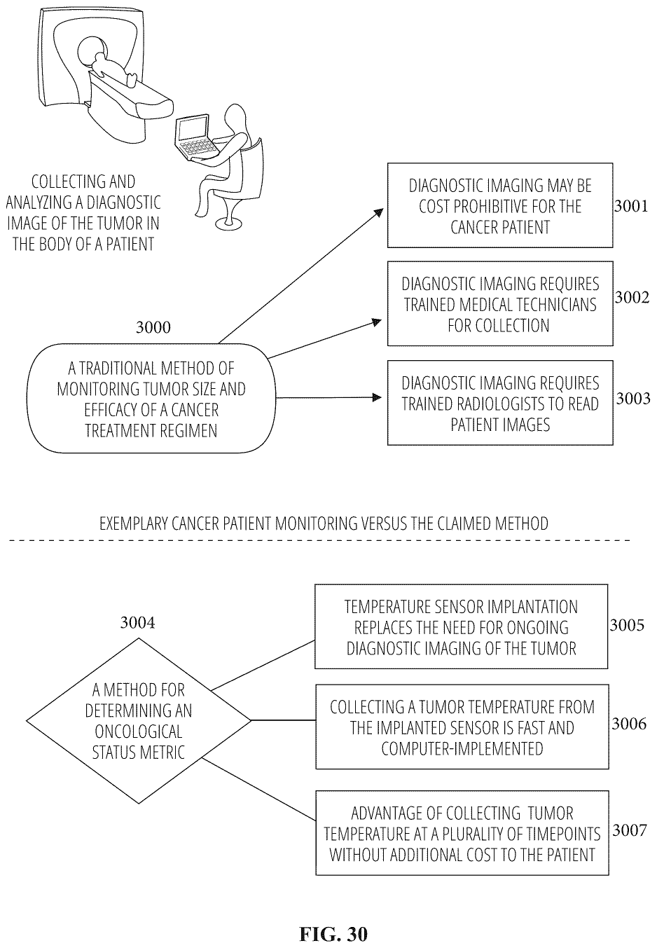

FIG. 30 compares a comparative method of monitoring cancer patients via diagnostic imaging of known tumors to the disclosed method of determining an oncological status metric. The disadvantages of the comparative approach to cancer monitoring and advantages of the disclosed method of cancer monitoring are illustrated.

FIG. 30 shows an example of a patient undergoing diagnostic medical imaging using an imaging device, such as using a computed tomography (CT) or magnetic resonance imaging (MRI) or other imaging modality, in order to monitor tumor size and efficacy of a cancer treatment regimen 3000. An imaging technician operates the imaging device to acquire diagnostic imaging data. Diagnostic imaging of a cancer patient can be expensive 3001, particularly if repeated over the course of treatment. Such diagnostic imaging can involve expense of an insurance co-pay associated with advanced imaging, of scheduling required to access imaging equipment and a trained imaging technician 3002, and of a trained medical radiologist to read and assess the collected diagnostic images 3003.

Comparatively, the present disclosed method of determining an oncological status metric 3004 can involve the one-time implantation of a temperature sensor 3005 at or near the patient's tumor for monitoring purposes. Once implanted, the temperature sensor may acquire temperature information at any time 3006, as directed by a user. Such monitoring using an implanted temperature sensor can be convenient for home-bound patients or patients with limited mobility. Tumor temperature measurements may be acquired as often or as infrequently as desired 3007, or according to the patient's plan of care. The temperature sensor can be configured to be capable of transmitting the collected temperature data to an external adjunct receiver device capable of receiving such temperature data. Thus, the patient's location need not impact the monitoring of the patient's cancer.

Costs associated with the disclosed method of determining an oncological status metric are primarily related to sensor implantation. Such costs do not accumulate according to the number of tumor temperature measurements acquired. This can help enable the patient's medical team to acquire data without excess cost to the patient or to the patient's health insurance provider. Furthermore, the implanted sensor may be configured to have the capability to collect physiologic data other than temperature data, such as one or more of a blood oxygen metric, a blood glucose metric or a blood pH metric. One or more such additional physiologic metrics collected from the implanted sensor may provide enhanced patient monitoring without increased medical cost.

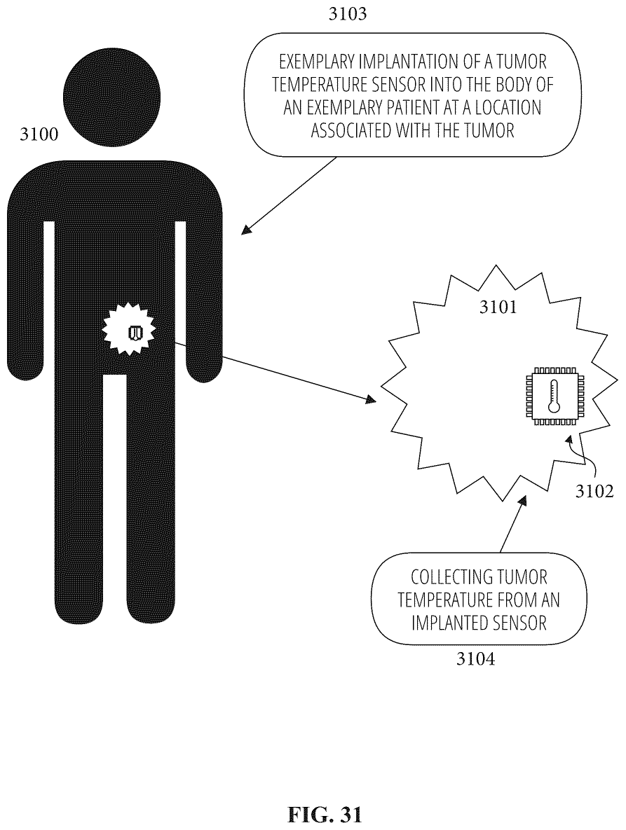

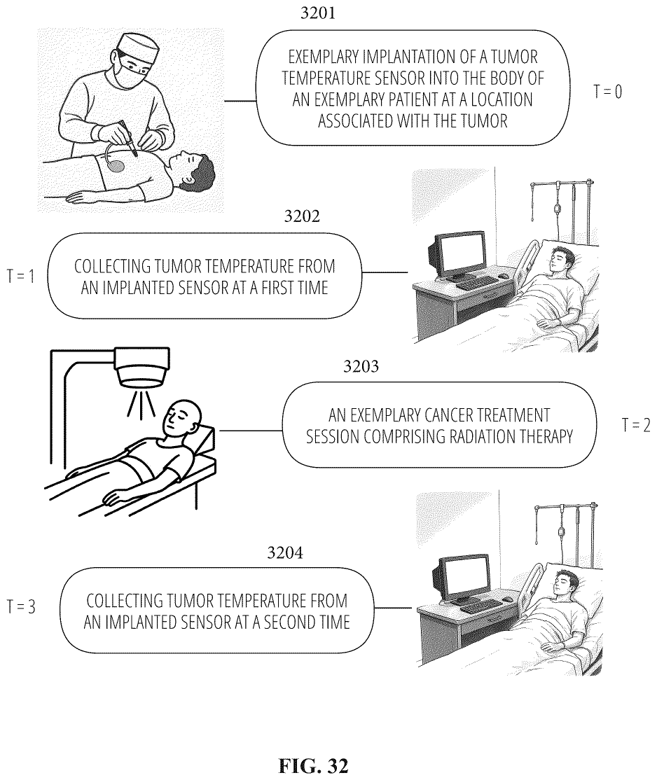

FIG. 31 illustrates an exemplary patient with at least one tumor in which an implantable sensor is located, such as which is capable of collecting and transmitting temperature information.

FIG. 31 illustrates an example of a cancer patient 3100 with a single tumor 3101 in which a temperature sensor 3102 can be implanted to collect tumor temperature data 3104. In a cancer patient in need of cancer monitoring, a physician or other caregiver can implant the temperature sensor in the body of the patient at a location that is closely associated with a known tumor 3103. The sensor may be implanted directly in the tumor tissue, nearby the tumor tissue, or locally adjacent to the tumor tissue. Optionally, a patient may have more than one sensor implanted. For example, a second sensor can be implanted in an association with an additional tumor or, alternatively, in a non-cancerous area of tissue in the patient's body. Where appropriate, only a single temperature sensor need be implanted, such as to help minimize the invasiveness of cancer monitoring and associated medical costs of implantation. The patient's medical team can consult with the patient on the implementation of the disclosed method, while taking into account the patient's medical history and current medical status.

FIG. 32 illustrates an exemplary timeline of events following the local implantation of a temperature sensor in an exemplary patient's tumor tissue 3201 (t=0), including the collection of tumor temperature measurements from the implanted sensor.

FIG. 32 illustrates an example of a timeline of events that may follow the implantation of the temperature sensor into a patient's body 3201. The sensor can be implanted in the least-invasive manner such as to help minimize patient distress and maintain the current health of the patient. After being implanted, the collection of temperature data can occurs automatically, without requiring further invasive surgical interventions. Passive or automatic collection of temperature data 3202 may include positioning the patient nearby a receiving device capable of receiving temperature data transmitted by the implanted sensor, such as by using a mutual-inductance-coupled telemetry, a Bluetooth or other radio-frequency (RF) telemetry, or the like. The distance between the transmitting temperature sensor and the receiving device may vary and may depend on the type of sensor implanted in the patient.

For example, the tumor temperature data may be collected at a first time 3202 (t=1 in FIG. 32), e.g., before a radiation therapy or other cancer treatment session 3203 (exemplified at t=2 in FIG. 32), and at a second time after the cancer treatment session 3204 (t=3 in FIG. 32). The timeline illustrated in FIG. 32 and is an exemplary timeline in which the disclosed method may be practiced. For instance, there may be a plurality (e.g., two or more) tumor temperature measurements collected before a cancer treatment session and a plurality (e.g., two or more measurements) collected after the cancer treatment session. In another example, there may be 3, 4, or 5 collected measurements before a cancer treatment session, such as from which a mean, median, or other central tendency metric can be computer, or only a single measurement. In another example, there may be a single temperature measurement collected after the cancer treatment session or 7 measurements collected at varying times after the cancer treatment session, such as between temporally adjacent treatment sessions, or with one or more other treatment sessions occurring interspersed with such measurements. The patient may undergo more than one cancer treatment session, and tumor temperature measurements may be collected at least once before and at least once after every cancer treatment session. The disclosed method of determining an oncological status metric for the patient is configurable to accept a minimum of two tumor temperature measurements or a plurality of measurements, which may depend on the patient's plan of care as informed by the patient's medical team.

The exemplary cancer treatment session depicted in FIG. 32 is radiation therapy, but the disclosed method to determine an oncological status metric may be practiced for patients undergoing other forms of cancer treatment, such as chemotherapy or targeted therapy. The disclosed method may be practiced over the course of multiple cancer treatment sessions or with respect to a single cancer treatment session. The accuracy of the disclosed method to determine the oncological status metric of a patient is not affected by the type of cancer treatment undergone by the patient. The use of the temperature sensor may be limited to the collecting temperature data before and after cancer treatment sessions, and the temperature sensor itself can be configured to be immune to the radiation therapy or other cancer treatments to which the patient is subjected.

One or more additional physiologic metrics may be collected from the patient, but they need not be collected from the implanted sensor, such as where other methods of collection are available. For example, the implanted sensor may only be capable of collecting temperature data and accordingly cannot be configured to collect the additional physiologic metrics, which can be collected from other patient monitoring devices or from an electronic medical record (EMR) associated with the patient. Collecting one or more additional physiologic metrics is not required to determine a patient's oncological status metric, but if available, they may be used to calculate or augment calculation of the oncological status metric.

One or more additional physiologic metrics, such as a blood oxygen metric, blood pH metric, or blood glucose metric may be collected at the same time instances at which tumor temperature is collected. This can help provide additional information regarding change in tumor physiology over the course of a cancer treatment regimen. For example, a decrease in blood oxygen near the tumor may indicate that vascular structures near or inside the tumor tissues are changing, or that current vasculature is insufficient to provide oxygen to a growing tumor (e.g., this may be a factor that can be used to calculate an indicated improvement in the oncological status metric). A decrease in blood pH near the tumor may indicate insufficient oxygenation to tumor tissues (e.g. this may be a factor that can be used to calculate an indicated improvement in the oncological status metric). An increase in blood glucose may indicate an increased stress response in response to either tumor growth or in response to the cancer treatment regimen (e.g. this may be a factor that can be used to calculate a change in the oncological status metric). The one or more additional physiologic metrics can supplement the collected tumor temperature data. This can enable determining the oncological status metric including considering multiple physiological changes to the patient, such as (but not limited to) one or more of acidosis, hypoxia, hyperglycemia, hypoglycemia, or cachexia.

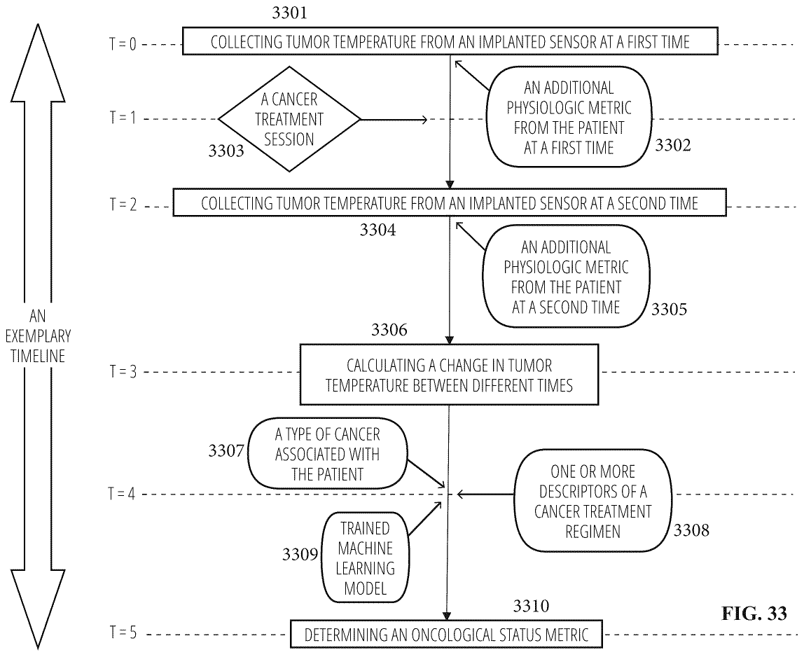

FIG. 33 is a schematic illustration of an exemplary timeline of events corresponding to the disclosed method for determining an oncological status metric.

At t=0, a tumor temperature measurement from an implanted sensor at a first time can be collected 3301, optionally together with one or more additional physiologic metrics 3302 such as can be used as factors in calculating the quantitative oncological status metric that can be used to infer efficacy of a patient's cancer treatment. For example, the tumor temperature can be collected at t=0 (3301) before a cancer treatment session occurs at t=1 (3303). At t=2 (3304), after the cancer treatment session, a second tumor temperature measurement can be collected from the sensor implanted in the patient, optionally together with one or more additional physiologic metrics 3305 such as can be used as factors in calculating the quantitative oncological status metric.

In FIG. 33, the steps of the disclosed method may be practiced at non-identical intervals. For instance, the time between collecting the second tumor temperature measurement (t=2) and then calculating the relative change in tumor temperature between timepoints t=0 (3301) and t=2 (3304) (e.g., occurring at t=3 (3306)), may be shorter than the time between the user's input of additional patient data at t=4, (e.g. cancer type 3307 and one or more descriptors of the cancer treatment regimen 3308), and the calculations at t=4, which can be performed using a programmed algorithm or using trained machine learning model 3309 at t=5 (3310) to calculate at the patient's oncological status metric.

The oncological status metric can be calculated at t=5 (3310), such as after the user's input of data (t=4) and the programmed algorithm or the trained machine learning model's processing of the newly acquired and inputted data (t=4). The calculated oncological status metric can vary based on one variable (e.g., tumor temperature) or multiple variables, such as described or incorporated herein. The time to input data relating to the current patient's cancer type and treatment regimen may depend on the availability of the disclosed method's user to enter such data. The time for the programmed algorithm or the trained machine learning model to process all of the newly acquired data, which can include two or more tumor temperature measurements by the implanted temperature sensor and the user-inputted details relating to the patient's cancer and treatment plan, may depend on both the size of the current patient dataset, as well as the data processing capabilities of the computing device used to perform the programmed algorithm or to employ the trained machine learning model's to calculate the oncological status metric.

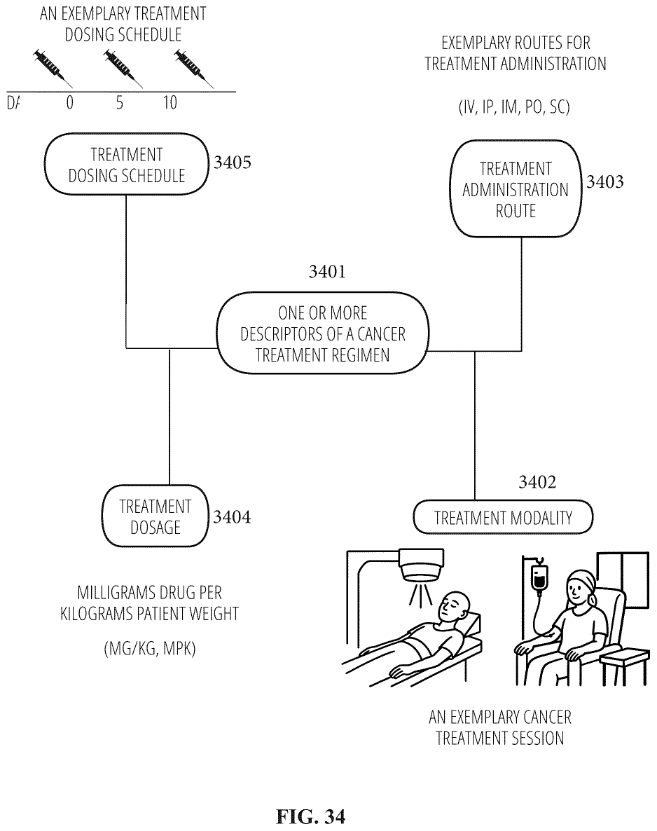

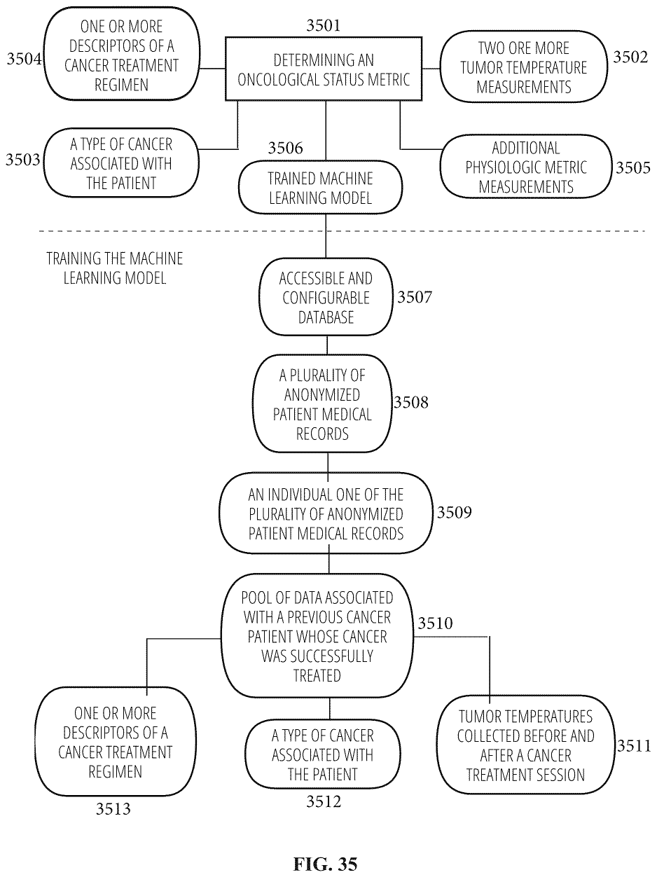

FIG. 34 is a schematic illustration of an example of one or more descriptors of a patient's cancer treatment regimen, such as can include the four types of data that may be included when describing an exemplary patient's cancer treatment regimen. For example, a treatment modality 3402 may include one or more of radiation therapy, chemotherapy, or targeted therapy. Targeted therapy is a field of precision medicine that targets one or more proteins that play a role in cancer cell growth, proliferation and/or metastasis. One or more drugs used for chemotherapy or targeted therapy may depend on, among other things, a patient's geographical location and may further depend on local laws or regulations to using certain drugs for treating cancer. Radiation therapy may not involve administering a drug, but some patients may undergo combination therapy, wherein radiation may sensitize the tumor tissue to effects of an administered drug. Targeted therapy and chemotherapy may be administered through different routes 3403, such as an intravenous (IV) route. Other routes for chemotherapy or targeted therapy administration can include intraperitoneal (IP), intramuscular (IM), oral (PO), or subcutaneous (SC) administration.

The dosage of a formulated drug for cancer treatment 3404 may depend on one or more factors, such as including, but not limited to, patient weight (e.g., milligrams of drug administered per kilograms of patient weight), drug potency, drug toxicity, and drug formulation. Furthermore, the treatment dosing schedule 3405 may depend on one or more factors including, but not limited to, the PK/PD (pharmacokinetics/pharmacodynamics) or ADME (adsorption/distribution/metabolism/excretion) properties of the drug being administered to the cancer patient.

FIG. 35 depicts an example of factors that can be used by the disclosed method to determine the oncological status metric 3501 for the patient currently being monitored. This can include three different sources of information that may be used in determining an oncological status metric for an exemplary patient, including the development of a trained machine learning model. For example, determining the oncological status metric can use at least two tumor temperature measurements 3502 collected from an implanted temperature sensor, such as to compute a deviation from a baseline. Additionally, determining the oncological status metric may employ patient data related to the type of cancer 3503 the patient is afflicted by, one or more descriptors of the cancer treatment regimen 3504 administered to the patient between the collection timepoints of the at least two tumor temperature measurements, as well as optional additional physiologic metric measurements 3505. A programmed algorithm or a trained machine learning model 3506 can be employed to compute the oncological status metric based on some or all of such factors.

FIG. 35 further shows an example of data on which a machine learning model can be trained, such as for determining the oncological status metric. A database of anonymized patient medical data is accessible to the machine learning model 3507 as least during training and may be configured to accept a single anonymized patient medical record 3509 or a plurality of anonymized patient medical records 3508. Each anonymized patient medical record can be associated with a pool of data 3510 that corresponds to a single, previous cancer patient, such as whose cancer was successfully treated. If available, the pool of data may include the type of cancer 3512 the previous patient was afflicted with before treatment, one or more descriptors of their cancer treatment regimen 3513, and at least two tumor temperatures collected from the implanted sensor, such as at timepoints before and after at least one cancer treatment session 3511.

Training of the machine learning model can be augmented as the dataset of previous, successfully-treated cancer patients grows in size (e.g., the number of anonymized patient medical records increases) or diversity (e.g., the dataset includes patient medical records corresponding to patients treated for a variety of cancer types and treated with a variety of cancer treatment regimens) or both of these. For example, the ongoing treatment of a patient suffering from breast cancer may be monitored by the disclosed techniques, such as for the purpose of determining an oncological status metric. While the trained machine learning model processes the newly acquired tumor temperature data, cancer-type data, and cancer treatment regimen descriptor data, it may refer back to acquired patient data from previous breast cancer patients whose breast cancer was treated successfully whilst tumor temperature data was collected.

As the data displayed in FIGS. 10A-10C illustrate, tumor temperature change occurring in patients treated for breast cancer, colon cancer and melanoma may not follow the same pattern. For example, the change in tumor temperature indicative of a successfully treated cancerous breast tumor may not be similar to the change in tumor temperature expected for a successfully treated cancerous colon tumor. The data presented from the in vivo tumor temperature monitoring study in mice suggest that successful treatment of breast cancer in mice may present as a tumor temperature decrease, while successful treatment of colon cancer in mice may not be accompanied by such a change in tumor temperature. Accordingly, the collection of tumor temperature data from implanted sensors in human patients afflicted by a particular type of cancer and subsequently successfully treated of the particular type of cancer will preferably be used in the programmed algorithm or to train a machine learning model, such that newly acquired tumor temperature data from a current human cancer patient can be processed by the programmed algorithm or the trained learning model to determine the patient's oncological status metric.

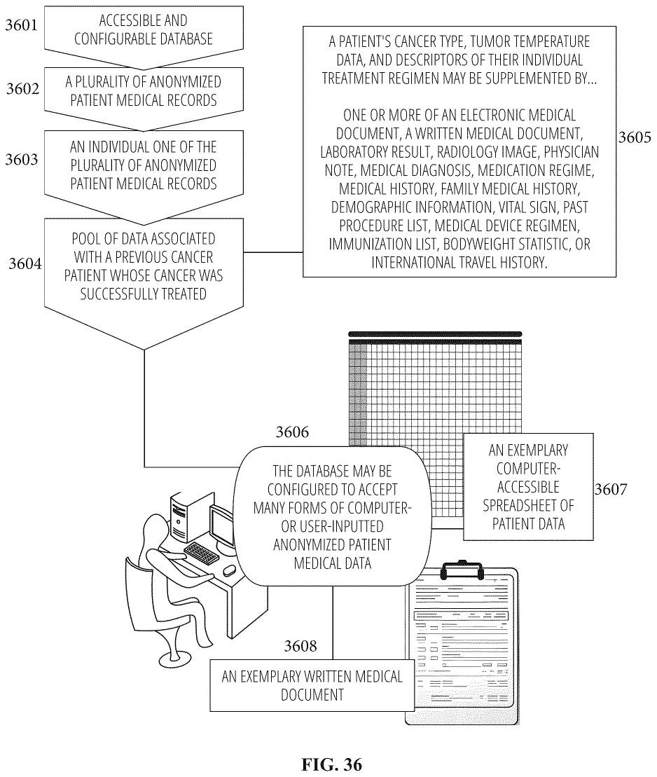

FIG. 36 is a schematic illustration of, among other things, the types of data that may be included in a database of anonymized patient medical records 3602, which is accessible to a machine learning model during training 3601.

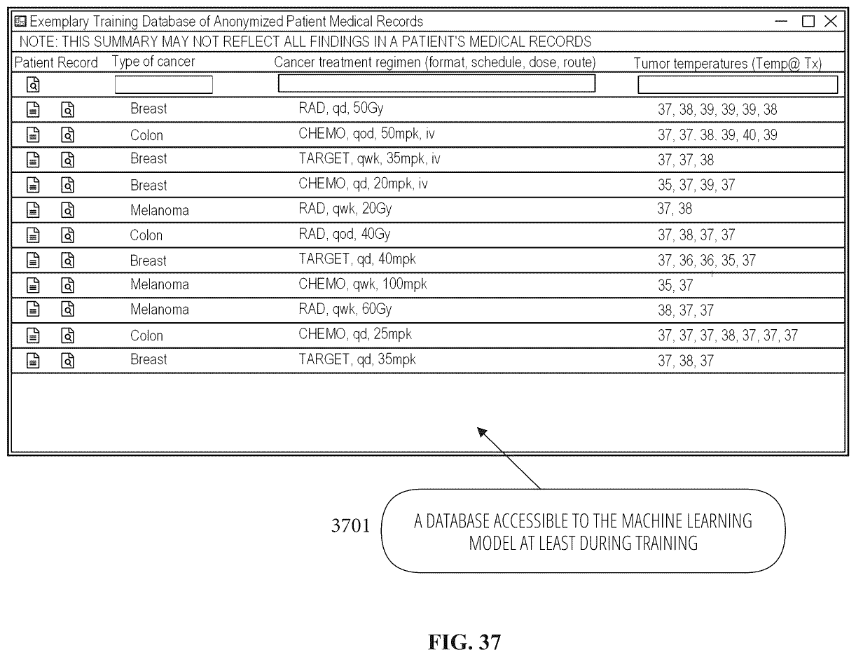

FIG. 36 further describes aspects of the database containing at least one anonymized patient medical record 3603 or a plurality of anonymized patient medical records 3602, such as which correspond to previous patients whose cancer was successfully treated 3604, which can be used to establish one or more parameters of the programmed algorithm or to train the machine learning model that will later process newly acquired patient data for determining an oncological status metric. Specifically, exemplary data formats that are acceptable 3606 to input into the database include exemplary written medical documents 3608 and exemplary computer-accessible spreadsheets of patient data 3607, along with various types of patient data that may be included in the pool of data associated with an individual one of the plurality of anonymized patient medical records. For example, one acceptable format of patient data is an exemplary computer-accessible spreadsheet of patient data, such as further described in FIG. 37.

Although none of the supplemental patient data exemplified in FIG. 36 is required for the present techniques of determining an oncological status metric, including such data in the database of anonymized patient medical records may help improve programming one or more parameters of an algorithm for determining the oncological status metric, or the training of the machine learning model, which may in turn improve the quality of inferences made by the trained machine learning model when processing new patient data for the purpose of determining a new oncological status metric. Supplemental data 3605 may include one or more of an electronic medical document, a written medical document, laboratory result, radiology image, physician note, medical diagnosis, medication regime, medical history, family medical history, demographic information, vital sign, past procedure list, medical device regimen, immunization list, bodyweight statistic, international travel history, or a blood pH metric, blood glucose metric, or blood oxygen metric collected from the previous cancer patient.

For example, supplemental data from previous cancer patients may be useful to train the machine learning model, such as to help make a more nuanced prediction of the oncological status metric. Additional physiological data about the previous cancer patient, such as laboratory tests or a medication regimen (not related to the cancer treatment regimen), may be used to teach the machine learning model that efficacy of the cancer treatment regimen may be affected by both generalized health metrics and medications unrelated to cancer treatment that may interact with one or more therapies used in the patient's cancer treatment regimen. Accordingly, the trained machine learning model use the current patient's supplemental data (e.g., one or more additional physiologic metrics) when evaluating cancer treatment efficacy and determining the oncological status metric. In another example, supplemental data such as a previous patient's family medical history may be used by a programmed algorithm or to train the machine learning model how familial genetic mutations can affect a cancer patient's response to one or more particular cancer therapies. Accordingly, the trained machine learning model can use similar supplemental data (e.g., one or more additional physiologic metrics) from current cancer patients when evaluating cancer treatment efficacy and determining the oncological status metric.

FIG. 37 illustrates user interface screenshot corresponding to an exemplary database of anonymized patient medical data 3701, which may include descriptors of patient cancer type, patient cancer treatment regimen, and tumor temperatures collected from each patient. The anonymized patient medical data would be accessible to the machine learning model during the training phase.

FIG. 37 provides an example of a spreadsheet or other modality of anonymized patient data, such as which may be included in the database of anonymized patient medical records, such as which can be accessible to the machine learning model during training. In this example of a dataset, three different types of cancer (e.g., breast cancer, colon cancer, and melanoma) are shown across multiple anonymized patient medical records. Three different formats of cancer treatment (e.g. treatment modalities such as radiation RAD, chemotherapy CHEMO, or targeted therapy TARGET) are shown. Also shown are various dosing schedules (e.g. qd, qod, qwk), numerous doses (in mg/kg or Gy), applicable routes of administration (e.g. intravenous), and all tumor temperature data collected over the course of the cancer treatment regimen (e.g. at least two discrete measurements occurring at two different timepoints).

The programming of a deterministic algorithm for computing the oncological status metric, or the training of the machine learning model may be enhanced by exposure to datasets with increasing complexity and increased diversity of data types. For example, a database including a plurality of cancer patient data in which a plurality of drugs were used, can be employed to train the machine learning model to predict how tumor temperature changes vary according to cancer treatment of a particular type of cancer with a specific drug type. For example, although the trained machine learning model may learn to generally predict how breast cancer tumor temperature responds to any type of drug treatment, including data corresponding to multiple drug types can help enable the trained machine learning model to predict whether the rate of tumor temperature change is correlated with drug treatment efficacy in the case of a particular drug type. In another example, a database including data from a plurality of melanoma patients in which a plurality of radiation doses were used, may be used to train the machine learning model to predict a rate of change in tumor temperature according to whether a relatively low or high dose of radiation is used during treatment.

For example, a trained machine learning model can process new patient data such that the change in tumor temperature can be analyzed relative to cancer type and treatment, but also the rate of tumor temperature change can be analyzed, such as it relates to the one or more descriptors of the cancer treatment regimen of the current patient. As the machine learning model is trained with increasing levels of data complexity, according to the database of previous cancer patient data, determining the oncological status metric can become a more nuanced prediction that considers a plurality of variables that may affect cancer treatment efficacy. The least complex calculation or prediction of an oncological status metric can be based only on tumor temperature data collected from implanted temperature sensors, while more complex calculation or prediction of an oncological status metric can be generated by the trained machine learning model, once the machine learning model has been trained on a sufficiently complex dataset of previous cancer patient data.

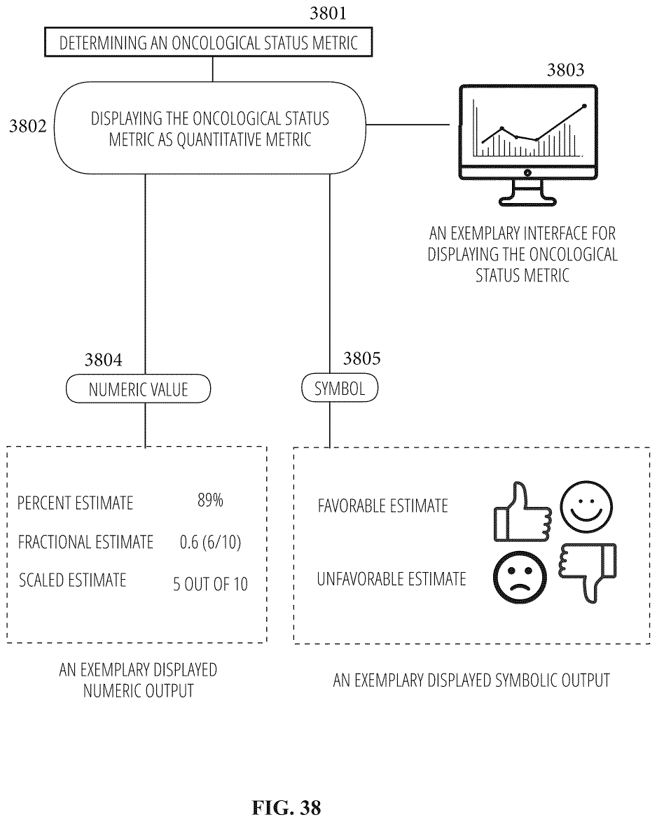

FIG. 38 is a schematic illustration of at least two exemplary ways in which an oncological status metric could be displayed to a user of the disclosed method for determining the oncological status metric.

FIG. 38 illustrates an example of determining the oncological status metric 3801, which can include displaying the oncological status metric to a user 3802. The oncological status metric is a quantitative metric 3802 that may be displayed to the user as a numeric value 3804 (e.g., such as a percent estimate, fractional estimate, or scaled estimate) or a non-numeric symbol 3805 (e.g., such as a thumbs up/down sign or a smiling/frowning face) that conveys the efficacy of the current cancer treatment regimen to the user. The interface on which the oncological status metric can be displayed 3803 may include any one or more of a number of devices with a digital screen, which can include, but not are not limited to, computer desktops, mobile phones, digital pagers, or digital tablets.

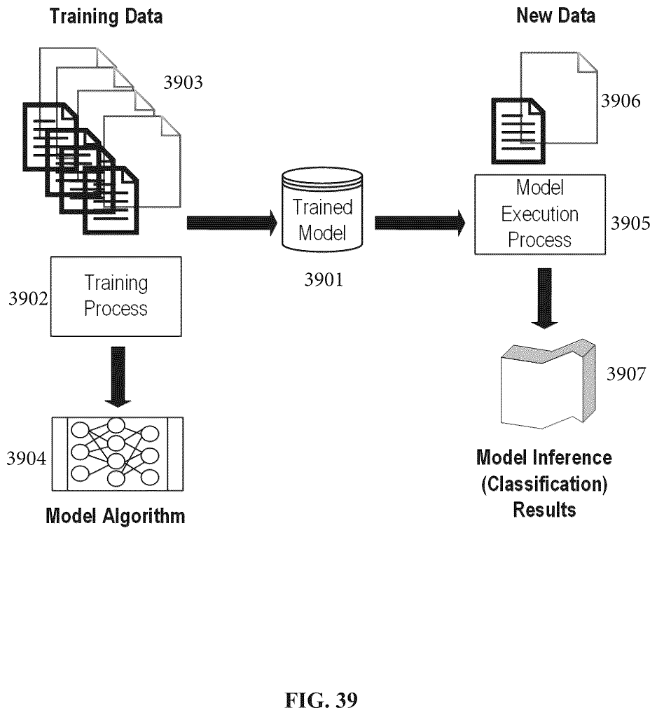

FIG. 39 is a schematic illustration of, among other things, an exemplary process by which a machine learning model is trained by an existing dataset prior to assessing new data.

FIG. 39 illustrates an example arrangement of a system that can be used to train, validate, and operate a machine learning model to classify data, generate inferences, perform regression, produce predictions or labels, or otherwise produce outputs from a data input. As shown, a trained machine learning model 3901 may be produced using a training process 3902. This training process 3902 may receive a set of training data 3903 that can be provided as input to the training process, to operate on a model algorithm to adjust the weights, values, or properties used within the model algorithm 3904 as part of a learning procedure. This model algorithm can involve unsupervised, supervised, or reinforcement learning approaches as part of training operations. In an example, the training data may include or be derived from image data, text data, data values, or some combination thereof. The model execution process 3905 is used to operate the machine learning model upon a set of new data 3906, such as data obtained for a current cancer patient. The model execution process 3905 is used to produce a model inference 3907 of a quantitative oncological status metric for the current patient, which may be displayed as a numeric value or non-numeric symbol.

While determining an oncological status metric need not require using diagnostic imaging data, the use of diagnostic imaging data from previous cancer patients whose cancer was successfully treated may be used during training of the machine learning model. The machine learning model may use the past diagnostic imaging data as a verification step during training, wherein a quantitative decrease in tumor size (as verified by tumor imaging studies) can be correlated to a corresponding change in tumor temperature over the course of the past patient's cancer treatment regimen.

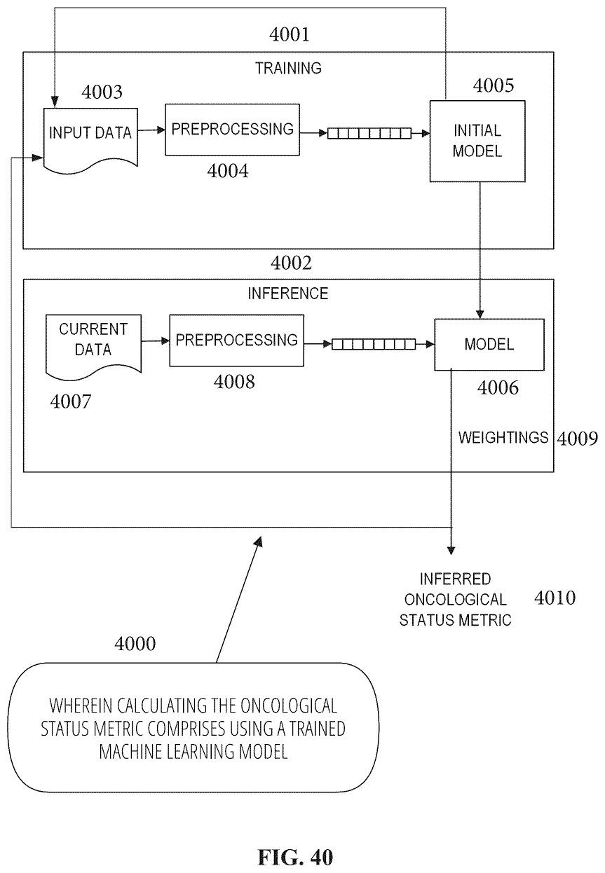

FIG. 40 is a schematic illustration of an exemplary process of training a machine learning model with previous cancer patient data in order to assess a current cancer patient's data and infer an appropriate oncological status metric 4000.

FIG. 40 illustrates an example of a machine learning model for training and execution related to determining an oncological status metric 4000 for a particular cancer patient. The machine learning model may be deployed to execute at a mobile device (e.g., a cell phone, a tablet, etc.) or a computer (e.g., a desktop, a laptop, etc.). The machine learning model can employ a training engine 4001 and an inference engine 4002. The training engine can use input data 4003 from previous cancer patients whose cancer was successfully treated, for example after undergoing preprocessing component 4004, to determine an oncological status metric.

The oncological status metric may be used to generate an initial model 4005, which may be updated iteratively or with subsequent labeled or unlabeled data (e.g., during reinforcement learning), for example to improve the performance of the inference engine or the initial model. An improved model 4006 may be redeployed for use. In the inference engine, current patient data 4007 may be input to preprocessing component 4008. The inference engine can produce a feature vector from the preprocessed current data, which is input into the improved model 4006 to generate one or more criteria weightings 4009. The criteria weightings 4009 may be used to output an inferred oncological status metric 4010, as discussed further below.

The training engine may operate in an offline manner to train the model (e.g., on a server connected to the database of anonymized patient data). The inference engine may be configured to operate in an online manner (e.g., in real-time, at a mobile device, on a wearable device, etc.). The model may be recurrently or periodically updated via additional training (e.g., via updated input data or based on labeled or unlabeled data output in the weightings) or based on identified later-acquired data, such as by using reinforcement learning to personalize a general model (e.g., the initial model) to a particular user.

The initial model may be updated using further input data until a satisfactory model is generated. The model generation may be stopped according to a specified criteria (e.g., after sufficient input data is used, such as 1,000, 10,000, 100,000 data points, etc.) or when data converges (e.g., similar inputs produce similar outputs). The specific machine learning algorithm used for the training engine may be selected from among many different potential supervised or unsupervised machine learning algorithms.

Examples of supervised learning algorithms include artificial neural networks, Bayesian networks, instance-based learning, support vector machines, decision trees (e.g., Iterative Dichotomiser 3, C9.5, Classification and Regression Tree (CART), Chi-squared Automatic Interaction Detector (CHAID), and the like), random forests, linear classifiers, quadratic classifiers, k-nearest neighbor, linear regression, logistic regression, and hidden Markov models.

Examples of unsupervised learning algorithms include expectation-maximization algorithms, vector quantization, and information bottleneck method. Unsupervised models may not have a training engine. In an example embodiment, a regression model is used and the model is a vector of coefficients corresponding to a learned importance for each of the features in the vector of features.

A reinforcement learning model may use Q-Learning, a deep Q network, a Monte Carlo technique including policy evaluation and policy improvement, a State-Action-Reward-State-Action (SARSA), a Deep Deterministic Policy Gradient (DDPG), or the like. Once trained, the model 420 may output an inference of the current patient's oncological status metric.

### EXAMPLES

### Example 1

### Introduction

Temperature changes in the body can be used as an indicator of disease. For cancerous tumors, localized temperature variations, both within the tumor and the body, can occur, such as can be due to changes in blood perfusion and metabolic heat generation. Cancer therapy can cause changes in energy balance dynamics within the tissue, resulting in a therapeutic response that can be monitored by tracking the temperatures.

Without being bound by theory, it is believed that tumor temperature change can be an early predictor of therapeutic response, such as in response to immunotherapy, before the changes in tumor size can be tracked by various imaging techniques. However, monitoring tumor treatment response reliably by measuring temperature can be challenging.

Physiological temperature can be measured via either a contact-based or contactless manner. Contact-based approaches (e.g., thermometer, thermistor, or thermocouples) can either be invasive or may only measure surface temperature. Non-contact approaches, such as thermography used in breast cancer screening, can only measure the skin temperature but cannot reliably measure temperature of the internal region of the tumor. Other imaging-based approaches, such as MR thermometry and photoacoustic thermometry, are either too expensive or too complicated to operate, making them not suited for monitoring deep body temperature over the time course of days to weeks. Therefore, a easy-to-use measurement system capable of providing real-time direct internal temperature is needed for long-term monitoring of disease progression and therapy response, especially in cancer.

Remote temperature monitoring can benefit both the patients and the healthcare system. To achieve this, reliable telemetric temperature sensors are needed. Implantable microchips that transmit temperature to an external transponder can be used for various purposes, including farm animals, pets, and experimental rodents. However, certain miniature wireless transponders are either too large to be used in tumors or may be subject to electromagnetic interference.

In this study, battery-free sensor devices can be used to transmit reliable temperatures and are small enough to be implanted into growing tumors on small animals in order to capture both basal and tumor temperature dynamics. Three preclinical models: melanoma (B16), breast cancer (4T1) and colon cancer (MC-38), and their corresponding cancer therapies: adoptive T cell transfer, AC-T chemotherapy, and anti-PD-1 immunotherapy, were used. Body and tumor temperature responses were recorded multiple times daily. Additionally, temperatures between the treatment and the control (no treatment) groups were compared.

This study provides the basis of relating the metabolic activity (represented by the temporospatial dynamics of the temperature) to tumor progression and cancer therapy. Our preclinical study suggests that high precision in vivo temperature monitoring can detect therapeutic responses by tracking tumor and body temperature changes following cancer treatment, especially immunotherapy. We postulate that changes in the temperature of the local tumor environment may be an early predictor and serve as response evaluation criteria in solid tumors (RECIST) for various forms of cancer therapy.

### Methodology

### In Vivo Temperature Measurement

All mice had 4-6 mm subcutaneous tumors placed in the right flank. Two sets of temperatures were collected: the basal temperature which reflects the body baseline and the tumor temperature which indicates the internal temperature of the tumor. The basal temperature was measured at the subcutaneous space on the left flank of the mouse using a temperature chip (2 mm diameter, 12 mm length) that transmits data to an external transponder at 400 kHz. The tumor temperature was measured by placing a temperature chip (1 mm diameter, 10 mm length) in the center of the tumor that transmits data at 134.2 kHz. Both chip types are built with two parts on both ends: the temperature sensing unit (made of thermistor and ASIC, respectively) and an RF transmitter/receiver unit. A thin (<0.3 mm) anti-migration sheath covering the temperature-sensing end was used to help immobilize the implants. No hindrance of animal mobility nor sign of discomfort following the chip implantation was observed.

Chip implantation was performed under general anesthesia with Ketamine/Xylazine. Sterile chips were loaded in a trocar and the skin's surface was prepped with 70% Ethanol. During the procedure, a small incision (˜2 mm, 5-10 mm away from the measurement side) was made to allow the chip to enter underneath the skin. The chips were then gently pushed with the temperature-sensing end facing front, below the dermis (left flank) or directly into the tumor (right flank). The incisions were then closed so that the chip stayed completely within the body.

Mice were individually caged during the temperature recording period. During the contactless measurements, animals were reached by placing the transponders below the cage without touching the cage to minimize disturbance to the animals. Information including the date, time, basal temperature, and tumor temperature was recorded. Mice were identified by both the cage number and the chip ID associated with the 134.2 kHz GTA chip.

### Cancer Models

The melanoma cell line (B16-F10) was obtained from ATCC. Cells were cultured in DMEM with 10% FBS and Pen-Strep. The TNBC cell line (4T1) was obtained from ATCC. Cells were in RPMI-1640 with 10% FBS and Pen-Strep. The colon adenocarcinoma cell line (MC-38) was obtained from Kerafast, provided by James W. Hodge and Jeffrey Schlom at National Cancer Institute. Cells were cultured in DMEM with 10% FBS, 2 mM glutamine, 0.1 mM nonessential amino acids, 1 mM sodium pyruvate, 10 mM HEPES, 50 μg mL-1 gentamicin sulfate, and Pen-Strep.

C57BL/6J mice (female, 8-10 weeks) were obtained from Jackson Laboratory (Bar Harbor, ME). BALB/c mice (female, 8-9 weeks) were obtained from Envigo. When >85% confluence was reached, the cells were detached by 0.05% Trypsin-EDTA and resuspended in phosphate-buffered saline in 20 million cells mL-1. Tumors were inoculated by injecting 50 μL of cell suspension subcutaneously into the hindlimb of mice. Experiments were performed 9-13 days after tumor seeding when a tumor diameter of 4-6 mm was obtained. Animals were randomized into the control group and the treatment group.

### Cancer Therapies

Adoptive T cells transfer for melanoma was achieved through intravenous (IV) transfer of TRP-2 T cells, which are CD8+ T cells bearing a genetically encoded high-affinity receptor against the TRP-2 tumor antigen from B16 tumor cells. TRP-2 specific T cells (at least 95% CD8+Thy1.1+) were isolated using a CD8+ T cell isolation kit. The TRP-2 T cells were further stimulated in vitro with IL-12 (2.5 ng/ml) and IL-2 (200 U/ml). 1 million activated TRP-2 T cells were transferred via IV to recipient mice through retro-orbital injection under general anesthesia with Isoflurane.

AC-T chemotherapy, which has been used clinically for treating breast cancer, for 4T1 tumor (TNBC model) was achieved by intratumoral injection of chemotherapeutic agents (0.1 ml) made up of Doxorubicin Hydrochloride (2 mg/kg), Cyclophosphamide (50 mg/kg), and Paclitaxel (5 mg/kg) solution in saline.

Anti-PD-1 immunotherapy, which has been approved for treating MSI-H and dMMR colorectal cancer, has also been demonstrated to be effective in preclinical colon cancer models including MC-38. Immunotherapy was delivered by intraperitoneal injecting 100 μg of antibody (In Vivo MAb anti-mouse PD-1, Clone RMP1-14 from Bio X Cell) on days 1, 3, and 5 on the mice bearing MC-38 tumor.

### Study Configuration

For each cancer model, a cohort of tumor-bearing mice following inoculation was divided into two groups: the treatment group and the control group. Cancer treatment was given to the treatment group only, as illustrated in FIG. 1A. Examples of experimental mice implanted with a pair of temperature chips for simultaneous recording of body temperature and tumor temperature are shown in FIG. 1B, 1C.

Mice were euthanized when the tumor reached endpoints (16 mm length or 2 cm³) or showed other signs of sickness or study-related complications, including skin ulceration. Data from the mice whose chip was found either dislodged from the tumor or not fully embedded under the skin were excluded from further analysis. The number of mice included in each group was no less than six.

### Data Processing and Statistics

Within each cancer model, for each mouse, two temperatures readings were recorded at time to: Tₜᵤₘₒᵣ(t0) and T_{body}(t0), and the difference in temperature between the tumor and the body is given by ΔT(t0)=Tₜᵤₘₒᵣ(t0)−T_{body}(t0). For comparison between two groups, ΔTA(t) or ΔTB (t) describes the average of ΔT within group A or group B. “t” is given in days, when t=0 represents the date of tumor inoculation (start of the tumor). Data are presented with average ±standard deviation.

Welch's t-test, using the two-tailed distribution and unequal variance, compares between two groups on the same day. P values were calculated to evaluate the significance of the difference between the two groups. P<0.05 is considered to be statistically significant.

### Results

Temperature Response of Melanoma Treated with Adoptive T Cell Transfer Immunotherapy

The B16-tumor-bearing mice had stable body and tumor temperature. The temperature chips were placed between day 14 and day 17 after the B16 tumor inoculation. In the control group, both the body and tumor temperature remained relatively steady over the recording period with daily temperature readings of 34.4±0.7 to 35.4±0.5° C. and 34.9±0.7 to 35.8±0.8° C., respectively, as shown in FIG. 2A between days 18 and 25, during which the tumor growth was unhindered before animals were euthanized.

The temperature response to adoptive T cell transfer was conspicuous with a decrease of both the body and the tumor temperature, following the immunotherapy on day 19, as shown in FIG. 2A. Body temperature started decreasing immediately following the immunotherapy, reaching the minimum on four days after the immunotherapy (day 23): 31.3±5.1° C. compared to 35.5±0.8° C. prior to the treatment. The body temperature difference of 2 groups (control vs immunotherapy) between days 21 and 24 was statistically significant (p=0.022, 0.004, 0.004 and 0.004). The tumor temperature also decreased after immunotherapy, with reading nadir on day 24 at 32.7±4.0° C. compared to 35.8±0.8° C. on day 18 before the therapy. The tumor temperature difference of the 2 groups (control vs immunotherapy) between days 22 and 24 was statistically significant (p=0.011, 0.002 and 0.009). Interestingly, for both measurements, the gap between the treatment and the control diminished on day 25.

Temperature Response of TNBC being Treated with Chemotherapy

In the 4T1 TNBC model, chips were implanted on day 18 following the inoculation. In the control group, the body temperature was stable till day 28, temperature ranging between 35.2±0.8 and 35.9±0.6° C., before starting to decrease to 34.2±1.5 by day 31, when the tumor burden started to take its toll, as shown in FIG. 2B. A steady decline of the tumor temperature was noticeable, from 35.5±0.6 to 32.1±1.9° C. towards the end of recording, as shown in FIG. 2B.

AC-T chemotherapy was given on days 23, 27, and 30. Chemotherapy caused no statistical differences (chemotherapy vs control) in either body or tumor temperature within the course of recording, as shown in FIG. 2B. Similar to that of the control group, the body temperature decreased between days 29 and 31, from 33.5±1.6 to 30.6±4.8° C. Chemotherapy did not reverse the downward trend of tumor temperature decline, even though the average tumor temperature briefly increased after the 1st and the 2nd dose of chemotherapy by 0.9-1.5° C. on days 23 and 27; however, the difference (chemotherapy vs control) was not statistically significant.

Temperature Response of Colon Cancer being Treated with Anti-PD-1 Immunotherapy

The body and tumor temperature of the MC-38 colon cancer model was relatively unchanged throughout the course of tumor growth. Chips were placed between days 11 and 14. In the control group, the body temperature stayed between 35.4±0.7 and 35.9±0.7° C., while the tumor temperature fell between 35.3±0.4 and 35.8±0.7° C., as shown in FIG. 2C.

Anti-PD-1 immunotherapy caused a significant reduction in body temperature but did not seem to affect tumor temperature. The first dose of antibodies was given on day 14 and the course of immunotherapy lasted till day 18. The body temperature decreased from 35.9±0.5° C. prior to treatment to 34.2±1.3° C. on day 22, as shown in FIG. 2C. The difference (control vs immunotherapy) was statistically significant on day 18 (p=0.026) and between days 21 and 23 (p=0.006, 0.027 and 0.035). The tumor temperature remained relatively unaffected, staying between 35.3±1.0 and 36.0±0.9° C., without a statistically significant difference from the control group, as shown in FIG. 2C.

### Temperature Difference Between the Tumor and the Body

As shown in FIG. 3A, B16 tumors consistently presented a higher temperature than the basal body temperature in this paired comparison. Despite a change in both body and tumor temperatures in response to the adaptive T-cell transfer, the gap between the two temperatures remained largely unchanged throughout the course of the tumor development and cancer treatment. In the control group, the tumor was 0.16±0.6° C. to 0.65±0.9° C. warmer than the body; while in the treatment group, the tumor remained 0.13±0.8° C. to 0.52±0.3° C. warmer than the body. The ΔT (tumor temperature minus body temperature) between the two groups was not statistically significant.

The evolution of ΔT in the 4T1 model is presented in FIG. 3B. The 4T1 tumor was cooler than the body, with a vast majority of recorded ΔTs being negative. In the control group, the ΔT was wider with the tumor progression: the ΔT decreased from −0.46±0.6 to −2.15±1.2° C. during the course of temperature monitoring. In the AC-T chemotherapy group, the descending trend of ΔT remained unchanged, corresponding to a drop of ΔT from 0.56±1.0° C. to −1.95±1.4° C. AC-T tends to slow the decrease of ΔT by a few days, as seen by an elevation of ΔT in the chemotherapy group compared to that of the control group on days 24-26 and 28-29, corresponding to 1-3 days after the 1st and 2nd dose of chemotherapy. The dips of ΔT on days 23 and 27 aligned with the IT administration of AC-T chemotherapy. Despite the variation of ΔT following chemotherapy, the differences were not statistically significant compared to those in the control group.

The distinctive feature of the ΔT in the MC-38 model with and without the anti-PD-1 immunotherapy is presented in FIG. 3C. The MC-38 tumor has a similar temperature to the body temperature yet slightly cooler than the body without any treatment. The ΔT remained mostly unchanged throughout the tumor progression, staying between −0.19±0.5° C. and 0.02±0.5° C. However, the ΔT is pronounced following the anti-PD-1 immunotherapy. In this group, the paired ΔT gradually increased from −0.02±0.5 to 1.86±1.3° C. The difference (control vs immunotherapy) was statistically significant between day 14 and day 23 (p=0.025, <0.001, 0.009, 0.014, 0.005, 0.004, 0.009, 0.005, 0.016 and 0.013). Given that the tumor temperature remained stable with or without the immunotherapy, the gap between the two temperatures was largely attributed to the decrease in body temperature in response to anti-PD-1 immunotherapy.

### Discussion

### Temperature Sensing and Therapeutic Response

The goal of this study was to provide basal and tumor temperature measurements in cancerous models that showed disease progression (control group) compared to those (treatment group) receiving their corresponding regimens (e.g., immunotherapy or chemotherapy) which are known to result in objective response in each of the models. We hypothesize that changes to in vivo temperature measurements of both the tumor bed and the basal body temperature can allow us to identify an early response to cancer therapies.

In this study, we found that tumor temperature can be similar to that of the body (such as MC-38), higher than body temperature (such as B16) or lower than the body temperature (such as 4T1). The change in temperature, ΔT, can stay relatively unchanged during tumor progression (such as B16 and MC-38), or become wider (such as 4T1, from an average of 0.56-1.95° C.) before the animals were overcame by the tumor burden.

Statistical differences in temperature can be observed as early as 1-2 days following the onset of immuno-therapy in our preclinical models. In the B16 model, the earliest significant differences between the treatment and the control group were observed to be 2 days (body temperature) and 3 days (tumor temperature) respectively. In the MC-38 model, a significant difference in ΔT was observed only 1 day following the first dose of anti-PD-1 antibody, a difference in body temperature when compared to the control groups was first observed on day 4 after the initiation of anti-PD-1 immunotherapy.

For comparison, tumor growth differences are either too small to differentiate or take a much longer time to show difference between groups, in the same model receiving the same treatment regimen. For instance, in the B16 model, it has been shown that TRP-2-specific T cells infiltrate the tumor but do not affect tumor growth. In the MC-38 model, it was shown that the difference in tumor growth is only observable after 13 days of anti-PD-1 immunotherapy.

Cancer angiogenesis and deregulated cellular energetics are hallmarks of cancer. “Thermal profiling” offers an additional dimension of cancer characteristics not available by conventional imaging and biopsy. The simple, low-cost, and non-invasive telemetric temperature sensing for monitoring cancer progression and treatment response can be a viable addition to existing tools for cancer diagnosis. Nevertheless, tumor response is likely multifactorial, and the level of response is on a continuum.

### Clinical Significance

Early detection of tumor response to therapeutic intervention has been a long-standing goal for physicians. Existing procedures of screening are either prohibitively resource-intensive and expensive or are unable to provide direct quantitative estimates of the relevant physiological parameters for accurate classification. As shown in this study and discussed herein, the measurable and statistically difference in temperature can precede the difference in tumor sizes in response to cancer immunotherapy, suggesting that the temperature response can be an early indicator of treatment response, without involving complex imaging or blood testing. The knowledge of tumor-type specific responses to particular drugs is clinically relevant for several reasons. For example, the pseudoprogression of immunotherapy can occur when the tumors grow larger following the therapy due to large amounts of tumor-infiltrating cells before eventually shrinking.

Being able to distinguish between an actual disease progression (not responding to immunotherapy) from pseudoprogression can help physicians make an informed decision on cancer management within a much shorter time frame, as compared to waiting for months by tracking changes in tumor volume. In addition, it may take a much longer time to determine the response of a solid tumor to immunotherapy than its response to other approaches, such as chemotherapy, surgery, or radiation therapy. However, from this study, the temperature responses can take place within a few days before changes in the size of the tumor. Being able to differentiate tumor response earlier can be both cost-saving and lifesaving. The present “telemetric thermal profiling” techniques can offer an inexpensive option to supplement established practice, therefore enabling physicians to make quicker and more informed decisions in cancer care.

One of the unique features of the temperature chips is that they can be FDA-approved to be permanently implanted into the tumor. After implantation, this technology enables on-demand monitoring of the tumor temperature that can be carried out at any time and on any frequency basis. This flexibility is helpful in a resource-limited setting, such as from the patient's home. The recorded temperature can be transmitted wirelessly to an external transponder without hindering the movement of the subject. This telemetric temperature sensing technique can facilitate the development of a cloud-based patient monitor system, therefore mitigating the laborious need for patients to present in person at a healthcare facility for tests and imaging.

The tumor temperature deviation from the body sheds light on the physiological features of the tumors. The tumor can have a temperature higher than body temperature, which can serve as the basis of infrared (IR) thermography for breast cancer screening as demonstrated for a handful of cancer types. However, the tumors can have a lower temperature than surrounding tissues in various preclinical models and clinical observations with Infrared thermography. In this study, the tumor temperature can be higher than body temperature, as shown with the B16 tumors, or lower than body temperature, as shown with MC-38. Moreover, the tumor progression changes the energy balance, therefore causing the tumor to shift from “hotter” to “colder” than the body, as shown in the 4T1 tumor model. The trend of tumors getting “colder” with the increase in volume is also noted in some preclinical models.

Non-invasive monitoring of body and tissue temperature is especially useful in thermal medicine. Thermal therapy, the manipulation of body or tissue temperature, has a broad medical application including cancer. For example, depending on the temperature and exposure time, heating can lead to direct cell death or can activate vascular, metabolic, and immunologic parameters of the tumor microenvironment, which may play an additional role in radiochemosensitization. Thermometry sensors can help in accurate evaluation of the quality of hyperthermia treatment and the calculation of the thermal dose delivered. Moreover, advanced heating systems can benefit from extensive thermometry such as for the effective utilization of temperature feedback power control.

### Potential Mechanism of Temperature Changes

The local temperature distribution and the energy balance within the tissue can be described by the Pennes bioheat transfer equation:

$$ρ {c}_{p} ( T ) \frac{\partial T}{\partial t} - ∇ ( k ( T ) ∇ T ) + {w}_{b} {c}_{b} ( T - {T}_{d} ) = {Q}_{met}$$

where T and t are temperature and time; ρ, cp and k are density, specific heat, and thermal conductivity of the biological tissues; ρb, c_{b}, w_{b}, Tₐ and Qₘₑₜ represent blood density, specific heat, perfusion rate, arterial temperature, and metabolic heat generation, respectively.

Between tumors and normal tissues, a distinctive difference in the metabolic heat generation and blood perfusion are the major factors affecting the bioheat transfer, while other thermal properties (density, specific heat, and thermal conductivity) are very similar to those of normal tissues (less than 10%, except for fat). Metabolic heat generation (Qₘₑₜ) of tumors can be 2.5×28 to 60×29 of that of normal tissue. Blood perfusion (w_{b}) can vary significantly depending on the tumor type and pathological conditions; it can increase with angiogenic increases in vascularity, leading to as much as 50× than that of normal tissue. However, necrotic tumors tend to have less blood perfusion due to tumor growth outpacing blood supply.

The pathophysiology of the tumor, either by tumor progression or external intervention, can also affect the tumor's thermal properties. For instance, the metabolic heat production (Qₘₑₜ) of a tumor is inversely proportional to the doubling volume time. In response to cancer therapy, there is higher heat generation (four-fold) by mitochondria during apoptosis compared to resting states, as well as a positive correlation between oxygen saturation and mitochondrial heating rate. The reduction of tumor temperature has been associated with necrosis (therefore reduced metabolism) accompanied by vascular disruption. Taken together, tumor temperature changes, both spatial and temporal, have significant diagnostic value by reflecting tumor physiology and response to treatment.

Systemic body temperature can also change with tumor progression and immunotherapy, even though the housing temperature of the implanted temperature sensor remains consistent. Body temperature changes can be reflected in Tₐ. There are some indications that the tumor-bearing mice “feel colder” than non-tumor-bearing mice. This is not well understood. The relation between metabolic stress associated with tumor growth and thermoregulation remains unclear. Moreover, mechanistic pathways linking metabolic cold stress and antitumor immunity are not yet defined. It may be that immunological defenses against tumors are energetically costly, therefore leading to the activation of thermogenesis.

In this study, based on our observation, tumor growth is accompanied with formation of necrosis core and angio-genesis at the outer edge of the tumors. B16 is the most necrotic among the three models. The B16 tumors are soft and fluid, while 4T1 tumors tend to be more solid and stiff. The 4T1 tumors present “pale” inner parts compared to “pink” edges, suggesting a lack of blood supplies to the center of the tumor. MC-38 is considered a “hot tumor,” characterized by high tumor mutational burden, increased expression of PD-L1 and IFN-γ signaling, and high T-cell infiltration, in contrast to “cold tumors” like B16.

In this study, no differences between tumor and body, or between control/treatment groups was observed in the chemotherapy-treated TNBC model. While cancer immunotherapy may involve tumor-filtrating lymphocytes (TILs) to be effective, chemotherapy drugs act on cancer cells directly. AC-T chemotherapy was administered intratumorally (other than IV) to increase the localized cytotoxic effect without increasing systemic toxicity. The lack of noticeable response for the AC-T regimen can be further investigated.

### SUMMARY

In this exemplary in vivo study in mice, temperature differentials (Tₜᵤₘₒᵣ−T_{body}) were calculated between healthy and tumor tissues in three oncology mouse models undergoing treatment for oncologic disease. The treatment comprised administration of a control substance, an immunotherapy, or a chemotherapy to the animal in need thereof.

In the B16 melanoma model, cancerous tissue demonstrates increased temperature in comparison to healthy tissue and regardless of treatment type. In the B16 melanoma model, animals were administered either a control treatment or an adoptive T cell transfer comprising activated TRP-2 T cells.

In the 4T1 breast cancer model, cancerous tissue demonstrates decreased temperature in comparison to healthy tissue and regardless of treatment type. In the 4T1 breast cancer model, animals were administered either a control treatment or an AC-T chemotherapy cocktail comprising doxorubicin hydrochloride, cyclophosphamide, and paclitaxel.

In the MC-38 colon cancer model, cancerous tissue demonstrates no difference in temperature compared to healthy tissue when control treatment is administered. However, administrating an immunotherapeutic triggers a local temperature increase in the cancerous tissue, which can be detected by the locally implanted temperature sensor device and is depicted as an increasing temperature differential over the course of treatment. In the MC-38 colon cancer model, animals were administered either a control treatment or anti-PD-1 immunotherapy comprising anti-PD-1 antibody.

In summary, this study provides the basis for monitoring temperature during tumor progression and the therapeutic response to chemotherapy and immunotherapies. This preclinical study suggests that high precision in vivo temperature monitoring can detect therapeutic responses to treatments by following tumor temperature changes during the treatment therapeutic window. Tracking of in vivo thermal activity was actualized with the precision and accuracy of the implanted devices, which offer earlier treatment assessment to patients without requiring complex imaging or lab testing.

On-demand monitoring of tumor temperature can at least be used to confirm treatment efficacy and adjust treatment regimen. Therefore, tumor telemetric temperature sensing can help facilitate a more efficient management plan and a reduction of patient burden. We posit that the inexpensive, accurate and telemetric temperature sensing has the promise of an accurate in situ screening and diagnostic approach for cancer management. Furthermore, the fundamental scientific premise of the present technique holds the potential of opening new vistas in rapid and affordable digital healthcare for early detection of tumor response.

### Example 2

### Introduction

Cancerous cells can cause temperature changes within their local and surrounding environment. This can be predominantly due to alterations in both metabolic rate and cellular perfusion. Changes in the temperature of the local cancer environment during treatment may occur much sooner than changes in tumor size. Therefore, it can be postulated that temperature changes in the local tumor environment can serve as early predictors of response for various forms of cancer therapy, including primary systemic therapy (neoadjuvant chemotherapy), immunotherapy, and radiation.

There are two methods of measuring physiological temperature: non-contact and direct contact. Non-contact methods, such as thermography used in cancer screening, measure temperature along the surface of the skin rather than within the local tumor environment. Certain contact-based methods, such as thermometers, can only measure temperature on the surface of the human body. Furthermore, other methods, such as MR thermometry and photoacoustic thermometry, can be either too costly or too complex, making them unsuitable for monitoring deep body temperatures over long periods. Therefore, a simpler and more reliable method for measuring the real-time temperature of the local cancer environment is needed to assess early responses to cancer treatment.

The present techniques for remote temperature monitoring in clinical settings can be performed using a telemetric temperature sensor. Similarly, this approach can be used in pets and farm animals using implantable microchips that transmit data to an external transponder. However, it should be noted that certain of these small wireless transponders are too large for use in tumor beds.

In this document, we utilized a battery-free microchip small enough to implant in a tumor bed to consistently transmit reliable measurements of temperature changes in both the tumor and the basal temperature of the organism. This was conducted using three cancerous animal models (melanoma B16, breast cancer 4T1, and colon cancer MC-38), which underwent treatment with adoptive T cell transfer, AC-T chemotherapy, and anti-PD-1 immunotherapy, respectively. These groups were compared to a control group that did not receive treatment. Tumor temperature and body temperature were measured multiple times per day in both the control and treatment groups. The goal of this study is to provide basal and tumor temperature measurements in cancerous models that exhibit disease progression and tumor size changes compared to those showing an objective response to their treatment regimen. We hypothesize that changes in in vivo temperature measurements of both the tumor bed and the basal body temperature can help identify an early response to cancer treatment regimens.

### Methodology

### Temperature Chip Placement and Measurements

All animals used in this study and the experimental procedure protocol were reviewed and approved by the Institutional Animal Care and Use Committee (IACUC) at the University of Minnesota. All mice had 4-6 mm subcutaneous tumors implanted on the right flank. Two sets of temperatures were collected: the basal temperature, which reflects the body baseline, and the tumor temperature, which indicates the internal temperature of the tumor bed. For each subject, the temperature measurement taken approximately 48 hours after implantation was used to determine an offset value between the two sensor types. This offset was used to adjust the 400 kHz (basal body) sensor to reflect a zero difference from the Geissler Temperature ASIC™ chip, which then allowed for detection of differences in measurements between sensors within subjects, reflective of any new changes after the initial 48 hours.

This offset technique can help facilitate using sensor repeatability levels as a valid limit of difference detection (delta). The offset was applied to all readings for each animal in determining temperature differences between tumor and body in serial measures. This technique can help mitigate differences between sensor device types and baseline differences between subjects, allowing for direct comparisons of group temperature deflections from the starting point based on treatment status. Appropriate central tendencies were applied after distributional analysis (e.g., median or mean).

The basal temperature was measured in the subcutaneous space on the left flank using a temperature chip that is 2 mm in diameter and 12 mm in length and transmits data to an external transponder at 400 kHz. The tumor temperature was measured by placing a Geissler Temperature ASIC™ chip, which is 1.5 mm in diameter and 10 mm in length, in the center of the tumor, transmitting data at 134.2 kHz. Both types of chips include two parts on each end: a temperature sensing unit made up of a thermistor and an application-specific integrated circuit (ASIC), and an RF transmitter/receiver unit. A thin (<0.3 mm) anti-migration sheath covering the temperature-sensing end was used to help immobilize the implants. No impairment of animal mobility or signs of discomfort after chip implantation were observed.

Chip implantation was performed under general anesthesia with ketamine/xylazine. Sterile chips were loaded into a trocar, and the skin surface was cleaned with 70% ethanol before the procedure. During the procedure, a small incision (˜2 mm, 5-10 mm away from the measurement side) was made to allow the chip to be placed under the skin. The chips were then gently pushed with the temperature-sensing end facing forward, below the dermis on the left flank or directly into the tumor on the right flank. The incisions were then closed to ensure that the chip remained completely within the body.

Mice were individually caged during the temperature recording period. During the contactless measurements, animals were reached by placing the transponders below the cage without touching it to minimize disturbance to the animals. Information including date, time, basal temperature, and tumor temperature was recorded. Mice were identified by cage number and the chip ID associated with the 134.2 kHz GTA chip.

A ±0.21° C. threshold was used as the minimum delta for valid detection differences in temperature calculations (between tumor and body) due to the 400 kHz sensor having a repeatability of ±0.20° C. and the GTA™ having a repeatability of ±0.0085° C. (rounded up to ±0.01° C.). Thus, 0.20° C.+0.01° C.=0.21° C. was used to discern valid difference detection levels for serial temperature monitoring within subjects.

### Cancer Model

The melanoma cell line (B16-F10) was obtained from the American Type Culture Collection (ATCC). Cells were cultured in Dulbecco's Modified Eagle Medium (DMEM) with 10% FBS and penicillin-streptomycin (Pen-Strep). The triple-negative breast cancer (TNBC) cell line (4T1) was also obtained from ATCC and cultured in Roswell Park Memorial Institute medium (RPMI-1640) with 10% FBS and Pen-Strep. The colon adenocarcinoma cell line (MC-38) was obtained from Kerafast, provided by James W. Hodge and Jeffrey Schlom at the National Cancer Institute. Cells were cultured in DMEM with 10% FBS, 2×10⁻³ m glutamine, 0.1×10⁻³ m nonessential amino acids, 1×10⁻³ m sodium pyruvate, 10×10⁻³ m HEPES, 50 μg mL⁻¹ gentamycin sulfate, and Pen-Strep.

C57BL/6J mice (female, 8-10 weeks) were obtained from Jackson Laboratory (Bar Harbor, ME). BALB/c mice (female, 8-9 weeks) were obtained from Envigo. When >85% confluence was reached, the cells were detached using 0.05% trypsin-EDTA and resuspended in phosphate-buffered saline at a concentration of 20 million cells mL⁻¹. Tumors were inoculated by injecting 50 μL of cell suspension subcutaneously into the hindlimb of the mice. Experiments were performed 9-13 days after tumor seeding when a tumor diameter of 4-6 mm was achieved. All animals were randomized into the control group or the treatment group.

### Cancer Therapy

Adoptive T cell transfer for melanoma was achieved through intravenous (IV) transfer of tyrosinase-related protein (TRP-2) T cells, which are cytotoxic (CD8) T cells bearing a genetically encoded high-affinity receptor against the TRP-2 tumor antigen from B16 tumor cells. TRP-2-specific T cells (at least 95% CD8+Thy1.1+) were isolated using a CD8 T cell isolation kit. The TRP-2 T cells were further stimulated in vitro with interleukin-12 (IL-12) (2.5 ng/mL) and IL-2 (200 U/mL). One million activated TRP-2 T cells were IV transferred to recipient mice through retro-orbital injection under general anesthesia with isoflurane.

Adriamycin and cyclophosphamide (AC-T) chemotherapy, which has been used clinically for treating breast cancer, for the 4T1 tumor (TNBC model) was administered by intra-tumoral injection of chemotherapeutic agents (0.1 mL), consisting of doxorubicin hydrochloride (2 mg/kg), cyclophosphamide (50 mg/kg), and paclitaxel (5 mg/kg) in saline.

Anti-programmed cell death protein 1 (anti-PD-1) immunotherapy, which has been approved for treating microsatellite instability-high (MSI-H) and mismatch repair-deficient (dMMR) colorectal cancer, has also been demonstrated to be effective in preclinical colon cancer models, including MC-38. Immunotherapy was delivered by intraperitoneal injection of 100 μg of antibody (In Vivo MAb anti-mouse PD-1, Clone RMP1-14 from Bio X Cell) on days 1, 3, and 5 for mice bearing MC-38 tumors.

### Study Configuration

For each cancer model, a cohort of mice was divided into two groups: the treatment group and the control group (the group that did not receive treatment). Mice were euthanized when the tumor reached endpoints (16 mm length or 2 cm³) or showed other signs of sickness or study-related complications, including skin ulceration. Data from mice whose chips were found either dislodged from the tumor or not fully embedded under the skin were excluded from further analysis. Post-mortem X-ray images were taken to visualize the placement of chips in the animals.

### Statistical Methodology

The analysis will begin with an examination of the distribution of tumor and body temperature readings using cumulative density function plots, along with an analysis of the differences between these measurements, focusing on treatment outcomes across different study groups. All analyses will only include readings that meet the inclusion criteria, starting from the first recorded pairing of tumor and body temperatures after the initiation of the treatment phase, with corresponding control groups analyzed alongside the treatment groups. The primary outcome measure will involve combining readings from two types of temperature sensors that have significantly different levels of measurement uncertainty.

To address these differences, the contribution of measurement errors to the overall variance in the data can be estimated. This can be achieved using a bootstrapping method that generates 20,000 simulated data sets, incorporating the uncertainty distributions of each sensor type. Using the estimated variances from these uncertainties, a Structured Equation Modeling (SEM) path analysis will be applied to adjust the data for measurement errors before calculating the primary outcome measures. An objective is to describe trends in the primary outcomes by treatment group within each study arm.

To evaluate treatment effects on the primary outcome measure, both non-parametric and parametric statistical methods are employed, selecting parametric tests when the data meet normality assumptions. Specifically a mixed model analysis of variance (ANOVA) will be used with random effects for individual mice, accounting for the influence of treatment group and time on the outcome measures. Time points will be recorded with minute-level precision and converted into treatment phase hours, ensuring detailed timing in our analysis. While the intervals between readings may vary, they will generally be regular on an 8-hour basis. If the mixed model indicates significant results, a radial smoother random effect model can be generated to further explore individual trends among subjects. This approach can help reduce noise and clarify how each subject's responses contribute to overall group effects.

In the radial smoother graphs, each reading can be categorized based on whether the body temperature was cooler by at least 0.3° C., within +0.3° C., or warmer by at least 0.3° C. compared to the baseline (day 1 of treatment). This labeling enables visualization of individual body temperature changes over time and identifies any significant deviations from baseline. Trends in these categories across treatment groups will be tested for using the Cochran-Armitage trend test.

### Inclusion/Exclusion Criteria

The data (3,610 body and tumor temperature readings) from all treatment arms were reviewed, and exclusion criteria were applied similarly. 776 (21.5%) temperature readings occurred before treatment start times and were excluded from the analysis. The primary outcome measurement used in this analysis was the temperature difference between the tumor sensor (134.2 kHz transponder) and the body sensor (400 kHz transponder). As such, only valid pairs where both tumor and body readings (taken within 1 minute of each other) were included. There were four (0.1%) measurements where the difference between tumor and body readings exceeded 6° C.; these were excluded from the analysis. This only occurred in the Melanoma study arm. Once either body or tumor temperature dropped below 30° C., the remaining data for that subject was excluded from the analysis. This impacted 90 (2.5%) readings in total. Any subject with less than six remaining valid readings was excluded from the analysis as some treatments did not reach peak activity within five successive readings. This excluded six (8.3%) subjects. The final inclusion count was 66 (91.7%) subjects with 2,668 (73.9%) readings.

### Primary Outcome Measure

The primary outcome measure for this analysis is the difference in temperature readings between the tumor and the body after applying an initial offset value to establish a baseline at the start of the treatment phase. This baseline, or setpoint, for each transponder can be determined from readings taken 48 hours after implantation, allowing any inflammation from the implantation procedure to subside. At the 48-hour mark, the recorded temperature for each sensor is used as the offset, effectively setting the reference point at 0° C. for all subjects and sensors. This ensures that all subjects start from the same reference point, allowing subsequent measures to track deviations from this setpoint to calculate the difference between tumor and body temperatures. This approach highlights the clinical relevance of this technology, as it allows for monitoring based on initial readings rather than absolute temperature values.

The formula for the primary outcome measure is as follows:

$$Primary Outcome Measure = [ Tumor ° C . ( @ Time = 48 hours + X ) - Tumor ° C . ( @ Time = 48 hours ) ] - [ Body ° C . ( @ Time = 48 hours + X ) - Body ° C . ( @ Time = 48 hours ) ] ;$$

$$Where X = time in the treatment phase .$$

All 132 setpoint readings (48 hours=0° C. difference) were excluded from the analyses. In this context, 48 hours represents the starting point of the treatment phase, and the repeated measures across time are relative to this treatment timeline. Using this offset and zeroing of tumor vs. body differences helps mitigate sensor accuracy, and placement differences prior to the treatment phase.

### Precision Differences Between Sensors

In this study, we used two types of transponder sensors to accommodate their proximity within the subjects. Both sensors are powered by an external reader via inductive coupling, allowing for a compact transponder design since all power is drawn from outside the subject. Due to the small size of the subjects, it was necessary for the transponders to operate at different coupling frequencies to ensure accurate readings from each sensor, as the inductive fields are larger than the subjects' bodies. The sensors used were a 400 kHz transponder from Bio Medic Data Systems (BMDS) and a 134.2 kHz Geissler Temperature ASIC (GTA™).

The GTA™, being the smaller of the two units, was chosen for implantation in the tumors across all three study arms. These temperature sensors utilize distinct technologies, resulting in significant differences in their precision and accuracy metrics. The GTA™ reports a temperature repeatability of +0.008° C., while BMDS indicates a repeatability of +0.20° C.

### Correction for Precision Differences

Measurement uncertainty can lead to regression dilution or attenuation bias, where the regression slope is underestimated or skewed toward zero due to measurement uncertainties rather than the actual effects being measured. In this study, systematic uncertainties inherent to each sensor type were taken into account, which vary significantly. Because paired data between sensor types is being analyzed, sensor differences are accounted for before conducting any computations. The reported repeatabilities can be used as a proxy to address sensor precision differences.

Structural equation modeling (SEM) offers a method for estimating the inherent variance associated with each sensor type's repeatability/precision, which addresses the random or non-systematic variance of interest from an experimental perspective. The SEM approach involves estimating the partial variance generated by sensor repeatability (uncertainty) errors. Each displayed temperature reading can be treated as a value on a distribution, with the most likely value centered around a normal distribution with a mean of 0 and a standard deviation (c) corresponding to the reported repeatability. By acknowledging the measurement uncertainties as proxied via reported sensor repeatabilities, the analysis can be enhanced, and this practice reduces regression attenuation bias by using these measurement repeatability error distributions.

For each study arm and treatment group, temperature values were assigned a random z-score from a Gaussian distribution with a mean of 0 and a standard deviation based on the sensor's repeatability. Each temperature reading was then adjusted to the center of the repeatability distribution as an estimate of its most probable or likely value, given its observed z-score assignment, resulting where the z-score of 0 falls within that distribution. Bootstrapping of the values obtained through z-scores was used to estimate the variance between observed and distribution centered values. These estimates are then applied to the SEM structure to approximate the impact each sensor's uncertainty has on the total variance of the data within the treatment group. The bootstrap resampling process was repeated 20,000 times for all 2,668 readings, with each replicate differing only in the random variations from the repeatability distribution. Finally, the variances for each treatment group and study arm (12 groups in total) were ranked from lowest to highest. The Interquartile Range (IQR) difference in uncertainty variance contribution (75th percentile-25th percentile) for each group was then used to estimate the average portion of variance attributable to repeatability errors. The SEM path analysis can be expressed as depicted in FIG. 4.

### Variance SEM Path Analysis

A partial variance structural equation modeling (SEM) path analysis was conducted, resulting in adjustments to the raw temperature values based on the model's estimated error contributions due to measurement uncertainty. Adjustments were not made if the estimated error was less than the reader's resolution threshold of ±0.1° C. However, if the adjustments exceeded this threshold, they were applied. The adjusted data was then utilized to establish the offset for the setpoints and to calculate the serial differences between the sensors.

The theoretical repeatability distribution graphs indicate that the 400 kHz sensor contributes significantly more uncertainty than the GTA™ sensor. In contrast, the GTA™ sensor retains its original reported temperature values.

The original observed and adjusted distributions, depicted with Kernel Density Estimation, illustrate how the variability-adjusted data maintains the characteristics of the observed data distribution, albeit with reduced variance. The Normal Density Function distributions clearly demonstrate that the central tendencies of the 400 kHz sensors increase due to the reduction in uncertainty variability.

### Results

### Melanoma and Immunotherapy Monitoring

The melanoma study arm included 25 subjects who met the inclusion criteria: 60% were in the control group, and 40% were treated with TRP-2 immunotherapy. The mean temperatures and 95% confidence intervals (CI) for the first seven treatment days show a generally increasing [tumor—body] temperature trend in the TRP-2 treated group, which was not observed in the control group. Cumulative distribution plots of temperature deflections from the setpoint show that the treatment group's body and tumor temperatures shifted leftward (cooler), with body temperatures showing a more pronounced shift than tumor temperatures (FIG. 11). Comparing the distribution of temperature differences [Tumor−Body] between the melanoma treatment groups using a non-parametric Kolmogorov-Smirnov Two-Sample Test yields a KSa of 2.6035 (p<0.0001), rejecting the null hypothesis that the treatment groups come from the same distribution. The [Tumor−Body] differences show a greater number of readings above 0° C. in the TRP-2 group, indicating that tumor temperatures exceeded body temperatures more frequently in this group than in the control group (FIG. 12).

A mixed-model repeated measures analysis, using subject (mouse) as a covariate, was performed to evaluate the interaction between treatment group and treatment time controlling for subject differences independent of the model main effects and their interactions. The covariate parameter (subject) was significant, with an estimate of 0.2873° C., Wald-Z=13.02, p<0.0001, indicating that subject differences were estimated to average 0.29° C. independent of treatment groups and treatment time or interactions. The intercept and treatment group effects were not significant. The interaction between treatment groups and treatment time was significant (F(2,339)=8.10, p=0.0004). The linear slope for the control group was estimated to be −0.00243° C./hour (95% CI=[−0.00403, −0.00084]), t(339)=−3.00, p=0.0029. The slope for the treatment group (TRP-2) was estimated to be 0.001967° C./hour (95% CI=[0.000523, 0.003411]), t(339)=2.68, p=0.0077. Statistical separation between the treatment groups was observed at 52 hours, with a temperature difference of 0.1904° C. (95% CI=[0.05354, 0.3272]) higher in the TRP-2 group compared to the control group's average (t(339)=2.74, p=0.0065) (FIG. 13).

Given the statistical significance of the covariate (subjects) a radial smoother (noise reduction technique) mixed model was performed that looked at subject changes over time. The interaction between treatment group and treatment time, with a random intercept, was tested using a type III fixed effect, yielding F(2, 194)=2.47, p=0.0869. The graph of this model shows, in general, positive slopes for individual subjects in the treatment group (TRP-2) and downward or flat slopes for the control group. For the treatment group, the positive slope trend is observed regardless of which sensor registers the warmer temperature. Body temperature change labeling, comparing body temperatures to baseline values on treatment day #1, visually suggests a difference between treatment groups. A categorical Cochran-Armitage trend test on the three body temperature ordinal categories in reference to treatment day #1 body temperature by treatment group shows a significant trend, with the treatment group having a higher percentage of cooler temperatures compared to day #1 of treatment (Z=−10.6372, p<0.0001) (FIG. 14).

### Breast Cancer and Chemotherapy Monitoring

The Breast Cancer study arm included 28 subjects who met the inclusion criteria. Of these, 57% were assigned to the control group and 43% received the AC-Taxol chemotherapy regimen. The control group remained eligible for inclusion nearly twice as long as the treatment group and accounted for 76% of the temperature readings. The mean temperatures for the first seven treatment days, along with the 95% confidence intervals (CI), show negative [tumor-body] temperature differences in the control group, which were not observed in the AC-Taxol treatment group. Cumulative distribution plots of temperature deflections from the setpoint visually show that the treatment group's body temperatures shifted leftward (cooler), while the tumor temperatures in the treatment group were comparable to those in the control group (FIG. 15).

Comparing the distribution of temperature differences [Tumor−Body] between Breast Cancer treatment groups using a non-parametric Kolmogorov-Smirnov Two-Sample Test yields a KSa of 2.3864 (p=0.0005), rejecting the null hypothesis that the treatment groups come from the same distribution. Visually, there is a rightward shift in the treatment group's cumulative distribution. Combined with the individual cumulative plots for Body and Tumor temperatures, this shift in the [Tumor−Body] difference is likely due to the cooler body temperatures in the AC-Taxol group (FIG. 16).

A mixed model repeated measures analysis using subject (mice) as a covariate was performed to evaluate the interaction between treatment group and treatment time. The covariate estimate (subject) was significant at 0.5028, Wald-Z=19.63, p<0.0001. The interaction between treatment groups and treatment time was also significant, F(2,771)=102.29, p<0.0001. The intercept and treatment group effects were not significant. The linear slope for the control group was −0.00328° C./hour (95% CI=[−0.00374, −0.00282]), t(771)=−14.09, p<0.0001. The slope for the AC-Taxol treatment group was −0.00222° C./hour (95% CI=[−0.00360, −0.00084]), t(771)=−3.16, p=0.0017. The estimated separation between treatment groups occurred at 62 hours of treatment time. At this time, the difference was 0.1825° C. (95% CI=[0.01460, 0.3504]) higher in the AC-Taxol group compared to the control group, t(771)=2.13, p=0.0332. Notably, the AC-Taxol group had ewer readings than the control group (374 versus 1176, respectively). As a result, the 95% confidence intervals for the AC-Taxol group are much smaller, which should be considered when comparing the regression slopes, as they are nearly parallel (FIG. 17).

A reduced noise model was employed in a mixed model with a random intercept and a random radial smoother, using mice as subjects. The overall interaction between treatment group and treatment time, with the random intercept, yielded a Type III fixed effect test of F(2,512)=8.91, p=0.0002. Notably, there was a group of mice whose tumor temperatures were at least 1° C. higher than their body temperatures. The body temperature status change, compared to body temperatures on treatment day #1, suggests a difference between the treatment groups. A Cochran-Armitage trend test revealed a significant trend: the treatment group showed a higher percentage of cooler temperatures compared to the baseline (treatment initiation day), Z=−6.3692, p<0.0001 (FIG. 18).

### Colon Cancer and Immunotherapy Monitoring

The Colon Cancer study arm included 13 subjects who met the inclusion criteria. Of these, 46.2% were assigned to the control group, and 53.8% were treated with anti-PD-1 immunotherapy. The temperature readings were distributed in proportion to the number of subjects in each treatment group.

The mean temperatures for the first seven treatment days, along with their 95% confidence intervals, show that the control group did not exhibit a linear trend during this period. In contrast, the anti-PD-1 treatment group demonstrated an upward trend over time.

Cumulative distribution plots of temperature deflections reveal no significant differences in Body or Tumor temperatures between treatment groups (FIG. 19). Comparing the distribution of temperature differences [Tumor−Body] between Colon CA treatment groups using a non-parametric Kolmogorov-Smirnov Two-Sample Test yields a KSa of 2.4805 (p=0.0002), rejecting the null hypothesis that the treatment groups come from the same distribution. A significant rightward shift is observed in the treatment group's cumulative distribution, particularly above the 80th percentile (FIG. 20).

A mixed model repeated measures analysis, using the subject (mice) as a covariate, was performed to evaluate the interaction between treatment group and treatment time. The covariate estimate for subject was significant at 0.3140, Wald-Z=10.30, p<0.0001. The intercept was also significant at −0.3193 (95% CI=[−0.5083, −0.1303]), t(212)=−3.33, p=0.0010. The treatment group effects were not significant. The interaction between treatment groups and treatment time was significant, F(2, 212)=20.10, p<0.0001. The linear slope for the control group was not significant(0.000779° C./hour, 95% CI=[−0.00080, 0.00236]), t(212)=0.97, p=0.3332. In contrast, the slope for the treatment group (anti-PD-1) was significant, with a value of 0.005260° C./hour (95% CI=[0.00361, 0.00691]), t(212)=6.27, p<0.0001. Treatment group separation was estimated to occur 81 hours into the treatment phase. At 81 hours, the difference in temperature was 0.1576° C. higher (95% CI=[0.00223, 0.3129]) in the anti-PD-1 group compared to the control group, t(212)=−2.0, p=0.0468. Notably, the anti-PD-1 group exhibited two distinct outcome measure clusters (FIG. 21).

A reduced noise model was applied in a mixed model with random intercept and random radial smoother, using mice as subjects. The overall interaction between treatment group and treatment time, with a random intercept, showed a Type III fixed effect test of F(2, 137)=1.56, p=0.2134. Notably, a group of mice exhibited an upward trend in outcome measures over time. Interestingly, for these mice, body temperatures had decreased compared to the treatment day #1 readings. The change in body temperature relative to day #1 did not indicate a significant difference between treatment groups. A Cochran-Armitage trend test did not reject the null hypothesis of equal proportions between treatment groups (Z=0.8006, p=0.4234) (FIG. 22).

### Discussion

This study explored the temperature dynamics of mice undergoing different treatment regimens, including TRP-2 immunotherapy, AC-Taxol chemotherapy, and anti-PD-1 immunotherapy, compared to control groups. The data suggest that while TRP-2 and AC-Taxol treatments induced temperature changes, the effects were more pronounced in the AC-Taxol and anti-PD-1 groups, with a significant difference in temperature trends over time. Notably, the AC-Taxol group showed an elevated tumor-body temperature distribution curve for the treatment group, likely due to lower body temperatures. The anti-PD-1 group exhibited an upward trend in tumor temperature, with treatment effects becoming significant after approximately 81 hours.

The differences in temperature dynamics between treatment groups support the notion that tumor and body temperatures may be useful indicators of therapeutic effects. The TRP-2 group showed body temperature shifts, but tumor temperature effects were less clear. This may reflect treatment-induced immune responses or inflammatory changes, suggesting that body temperature could serve as a proxy for immune activity. The AC-Taxol group's cooler body temperatures may reflect a chemotherapy-induced systemic effect, but the lack of significant tumor temperature changes raises the question of whether these treatments are targeting the tumor directly or inducing the expected thermal responses in the tumor microenvironment. Anti-PD-1 treatment, on the other hand, demonstrated a notable upward trend in tumor temperatures, indicating that immune checkpoint inhibition might alter the tumor microenvironment. However, the presence of two distinct outcome measure clusters in this group suggests heterogeneity in treatment responses.

The ability to detect changes in tumor temperature can also be studied in the context of monitoring response to thermotherapy. Temperature change as an objective measurement of response to therapy is a point of focus for the present techniques. While the exact pathophysiology of how changes in temperature reflect underlying molecular processes is not fully understood, there are several theories. For example, tumor metabolic activity can affect thermal properties, with the metabolic heat production of a tumor being inversely proportional to its doubling volume time. Furthermore, tumor temperature can change in response to treatment. In particular, mitochondrial heat generation during apoptosis is four times higher than in the resting state.

Additionally, a reduction in tumor temperature can be linked to tumor necrosis and vascular disruption, often resulting from a decrease in metabolism within the tumor microenvironment. There can be a positive correlation between oxygen saturation and mitochondrial heating rate in tumor tissue. Thus temperature changes may reflect metabolic and vascular alterations within the tumor, which could provide valuable insights into treatment response, as seen in our study.

A key strength of this study is the careful correction for sensor precision differences. Both sensor types (400 kHz and 134.2 kHz) had varying repeatability, and the use of bootstrapping and structural equation modeling (SEM) to estimate and correct for measurement uncertainty helped mitigate the risk of regression attenuation bias. These methodological advancements enabled accurate estimates of temperature trends and avoided misleading interpretations due to sensor errors.

The ability to detect tumor response to cancer therapy has been a longstanding goal in oncology. Other approaches of assessing treatment response may rely on methods that are often time-consuming, expensive, and invasive. For instance, carcinoembryonic antigen (CEA) can be a tumor biomarker in colorectal cancer (CRC) but suffers from limitations such as variability in specificity and sensitivity, and differences in CEA levels among individual patients. Similarly, imaging with 18F-fluorodeoxyglucose (FDG) or other radiotracers used in PET/CT scans is another method for monitoring treatment responses in some cancers, but these techniques involve recurrent radiation exposure and require patients to visit imaging centers, making them less convenient.

The variety of options for measuring treatment response is a result of the diverse nature of cancer itself. Different cancers may involve distinct tissues, which can influence which diagnostic methods are most appropriate for monitoring treatment progress. However, changes in temperature within the tumor microenvironment occur across all types of cancer, as they all exhibit some degree of metabolic activity associated with neoplasia. This presents an exciting opportunity for the present approach using temperature-sensing technologies—such as the devices used in this study—to potentially serve as a standardized, non-invasive monitoring tool for all treatable cancers. By continuously measuring temperature changes, these technologies can help provide real-time, objective data to assess tumor response to treatment, thus reducing the reliance on invasive and often costly procedures.

The ability for patients to undergo treatment and remote monitoring without the need for repeated hospital visits significantly reduces the logistical burden on patients, especially for those who experience difficulties in traveling for follow-up appointments. Transportation from home to healthcare centers is a social determinant of health, and challenges in accessing care contribute to treatment delays or interruptions for thousands of patients.

Remote temperature monitoring addresses this social barrier by providing a low-cost, low-stress method for patients to engage with their treatment while remaining at home. By reducing the frequency of in-person visits, the present approach may lead to improved treatment adherence and outcomes.

This study provides valuable insights into the potential role of temperature dynamics as a biomarker for treatment efficacy and contributes to a growing body of evidence suggesting that temperature dynamics could serve as a non-invasive, real-time biomarker for monitoring treatment efficacy. This approach helps clinicians assess therapy responses more quickly and accurately, leading to better patient outcomes and personalized treatment strategies. The study underscores the potential of temperature-sensing technologies as an emerging tool in oncology, capable of offering a cost-effective, low-risk, and reliable method to monitor tumor response across a wide range of cancers.

What is claimed is:

1. A method to determine an oncological status metric in a patient, the method comprising:
collecting a tumor temperature from an implanted sensor, located in association with a tumor in a body of the patient, the collecting occurring at different times and including recurrently collecting at one or more times after one or more cancer treatments; and
calculating the oncological status metric, using processor circuitry, based on a change in the tumor temperature collected at the different times including the one or more times after one or more cancer treatment.

2. The method of claim 1, wherein the collecting uses the implanted sensor located in the tumor in the body of the patient.

3. The method of claim 1, further comprising:
collecting an additional physiologic metric including one or more of a blood pH metric, a blood glucose metric, or a blood oxygen metric from the patient; and
also using the additional physiologic metric for calculating the oncological status metric.

4. The method of claim 3, wherein at least one of the collecting the tumor temperature or the collecting the additional physiologic metric occurs both before and recurrently after a cancer treatment session.

5. The method of claim 1, wherein calculating the oncological status metric further comprises calculating based on a type of cancer associated with the patient.

6. The method of claim 1, wherein calculating the oncological status metric is carried out without requiring collecting and analyzing a diagnostic image of the tumor in the body of the patient.

7. The method of claim 1, wherein calculating the oncological status metric comprises using a programmed algorithm or a trained machine learning model.

8. The method of claim 7, wherein the machine learning model is trained using a plurality of anonymized patient medical records.

9. The method of claim 8, wherein an individual one of the anonymized patient medical records corresponds to a previous cancer patient whose oncological status metric was calculated before and after a cancer treatment session.

10. The method of claim 9, wherein the individual one of the anonymized patient medical records corresponds to the previous cancer patient with a tumor in which an implanted sensor was implanted to provide a tumor temperature.

11. The method of claim 8, wherein an individual one of the anonymized patient medical record comprises a pool of data associated with a previous cancer patient whose cancer was successfully treated.

12. The method of claim 11, wherein the pool of data associated with the previous cancer patient whose cancer was successfully treated comprises the tumor temperature collected before and after a cancer treatment session and one or more descriptors of a cancer treatment regimen.

13. The method of claim 12, wherein the one or more descriptors of the cancer treatment regimen comprise one or more of a treatment modality, treatment dosage, treatment administration route, and treatment dosing schedule.

14. The method of claim 8, wherein the plurality of anonymized patient medical records are included in a database accessible to the machine learning model at least during training.

15. The method of claim 14, wherein a size of the database is configurable to accept one or more additional anonymized patient medical records.

16. The method of claim 11, wherein the pool of data associated with the previous cancer patient whose cancer was successfully treated further comprises one or more of a blood pH metric, blood glucose metric, or blood oxygen metric collected from the previous cancer patient.

17. The method of claim 1, wherein the oncological status metric includes a quantitative metric capable of being displayed or otherwise output as a numeric value or symbol.

18. The method of claim 4, wherein the cancer treatment includes one or more sessions of radiation, chemotherapy, immunotherapy, and/or targeted therapy.

19. The method claim 11, wherein the pool of data associated with the previous cancer patient whose cancer was successfully treated further comprises one or more of an electronic medical document, a written medical document, laboratory result, radiology image, physician note, medical diagnosis, medication regime, medical history, family medical history, demographic information, vital sign, past procedure list, medical device regimen, immunization list, bodyweight statistic, or international travel history.

20. A method to determine an oncological status metric in a patient, the method comprising:
(1) collecting, wherein collecting comprises:
(a) collecting a tumor temperature from an implanted sensor, located in association with a tumor in a body of a patient;
(b) collecting uses the implanted sensor located in the tumor in a body of the patient;
(c) collecting an additional physiologic metric including one or more of a blood pH metric, a blood glucose metric, or a blood oxygen metric from the patient; and
(d) at least one of the collecting the tumor temperature or the collecting the additional physiologic metric occurs both before and after a cancer treatment session; and
wherein the cancer treatment includes one or more sessions of radiation, chemotherapy, immunotherapy, and/or targeted therapy; and
(2) calculating, wherein calculating comprises:
(a) calculating the oncological status metric, based on a change in the tumor temperature collected at different times;
(b) calculating based on a type of cancer associated with the patient;
(c) calculating using the additional physiologic metric for calculating the oncological status metric;
wherein the oncological status metric includes a quantitative metric capable of being displayed or otherwise output as a numeric value or symbol;
(d) calculating the oncological status metric includes calculating without requiring collecting and analyzing a diagnostic image of the tumor in the body of the patient; and
(e) calculating the oncological status metric comprises using programmed algorithm or a trained machine learning model;
wherein the machine learning model is trained using a plurality of anonymized patient medical records;
wherein the plurality of anonymized patient medical records are included in a database accessible to the machine learning model at least during training;
wherein the database is configurable to accept one or more additional anonymized patient medical records;
wherein an individual one of the anonymized patient medical records corresponds to a previous cancer patient whose oncological status metric was calculated before and after a cancer treatment session;
wherein the individual one of the anonymized patient medical records corresponds to the previous cancer patient with a tumor in which an implanted sensor was implanted to provide a tumor temperature;
wherein an individual one of the anonymized patient medical record comprises a pool of data associated with a previous cancer patient whose cancer was successfully treated; and
wherein the pool of data associated with the previous cancer patient whose cancer was successfully treated comprises the tumor temperature collected before and after a cancer treatment session and one or more descriptors of a cancer treatment regimen;
wherein the pool of data associated with the previous cancer patient whose cancer was successfully treated further comprises one or more of an electronic medical document, a written medical document, laboratory result, radiology image, physician note, medical diagnosis, medication regime, medical history, family medical history, demographic information, vital sign, past procedure list, medical device regimen, immunization list, bodyweight statistic, international travel history, or a blood pH metric, blood glucose metric, or blood oxygen metric collected from the previous cancer patient; and
wherein the one or more descriptors of the cancer treatment regimen comprise one or more of a treatment modality, treatment dosage, treatment administration route, and treatment dosing schedule.
